SEQUENCE LISTING (54 sequences)

SEQ ID NO: 1 - DNA, 74 bp, Artificial Sequence
  Description of Artificial Sequence Synthetic primer
ataacaacacgcaacatggatgtccacgaggtctcttactggacggcacggtttatcgtacgctgcaggtcgac

SEQ ID NO: 2 - DNA, 74 bp, Artificial Sequence
  Description of Artificial Sequence Synthetic primer
tagcaaactcttaaactacggtgtcggtctcgtagaacggttgctaatgtttccgatcgatgaattcgagctcg

SEQ ID NO: 3 - DNA, 18 bp, Artificial Sequence
  Description of Artificial Sequence Synthetic primer
gatgtccacgaggtctct

SEQ ID NO: 4 - DNA, 17 bp, Artificial Sequence
  Description of Artificial Sequence Synthetic primer
cggtgtcggtctcgtag

SEQ ID NO: 5 - DNA, 18 bp, Artificial Sequence
  Description of Artificial Sequence Synthetic primer
cgtacgctgcaggtcgac

SEQ ID NO: 6 - DNA, 19 bp, Artificial Sequence
  Description of Artificial Sequence Synthetic primer
atcgatgaattcgagctcg

SEQ ID NO: 7 - DNA, 45 bp, Artificial Sequence
  Description of Artificial Sequence Synthetic primer
agtacgttaaaatcaggtatcaagtgaataacaacacgcaacatg

SEQ ID NO: 8 - DNA, 45 bp, Artificial Sequence
  Description of Artificial Sequence Synthetic primer
aaaaaaaaaaacgggagattaaccgaatagcaaactcttaaacta

SEQ ID NO: 9 - DNA, 25 bp, Artificial Sequence
  Description of Artificial Sequence Synthetic primer
gataagattgcgacaaaagaggata

SEQ ID NO: 10 - DNA, 25 bp, Artificial Sequence
  Description of Artificial Sequence Synthetic primer
cagtatggaactgacacatttcttg

SEQ ID NO: 11 - DNA, 25 bp, Artificial Sequence
  Description of Artificial Sequence Synthetic primer
ctagtgttcagttgaatcacacacc

SEQ ID NO: 12 - DNA, 25 bp, Artificial Sequence
  Description of Artificial Sequence Synthetic primer
gtgggacgagacgtttagatagtaa

SEQ ID NO: 13 - DNA, 45 bp, Artificial Sequence
  Description of Artificial Sequence Synthetic primer
gccatacaaaacattcgtggctacaactcgatatccgtgcagatg

SEQ ID NO: 14 - DNA, 45 bp, Artificial Sequence
  Description of Artificial Sequence Synthetic primer
tttcacattatttatattcgtatatacttccaactctttttttca

SEQ ID NO: 15 - DNA, 74 bp, Artificial Sequence
  Description of Artificial Sequence Synthetic primer
tcgatatccgtgcagatggatgtccacgaggtctctattgcattcgcacttccgatcgtacgctgcaggtcgac

SEQ ID NO: 16 - DNA, 74 bp, Artificial Sequence
  Description of Artificial Sequence Synthetic primer
ttccaactctttttttcacggtgtcggtctcgtagtatgatcggacaccacgcagatcgatgaattcgagctcg

SEQ ID NO: 17 - DNA, 25 bp, Artificial Sequence
  Description of Artificial Sequence Synthetic primer
atgctttttcgtagctaacttctca

SEQ ID NO: 18 - DNA, 25 bp, Artificial Sequence
  Description of Artificial Sequence Synthetic primer
tggtcttacgacagctagagagttt

SEQ ID NO: 19 - DNA, 25 bp, Artificial Sequence
  Description of Artificial Sequence Synthetic primer
attactcaggaatgattacatccca

SEQ ID NO: 20 - DNA, 25 bp, Artificial Sequence
  Description of Artificial Sequence Synthetic primer
aagtgagtatttggctcaacagaac

SEQ ID NO: 21 - DNA, 45 bp, Artificial Sequence
  Description of Artificial Sequence Synthetic primer
aaagggaaagttgagcactgtaatacgccgaacagattaagcatg

SEQ ID NO: 22 - DNA, 45 bp, Artificial Sequence
  Description of Artificial Sequence Synthetic primer
atgaaggttgccgaaaaaaaattattaatggccagtttttcctca

SEQ ID NO: 23 - DNA, 74 bp, Artificial Sequence
  Description of Artificial Sequence Synthetic primer
ccgaacagattaagcatggatgtccacgaggtctctagagtcatcccattacctagcgtacgctgcaggtcgac

SEQ ID NO: 24 - DNA, 74 bp, Artificial Sequence
  Description of Artificial Sequence Synthetic primer
atggccagtttttcctcacggtgtcggtctcgtagtaggctaagagttgctaacgatcgatgaattcgagctcg

SEQ ID NO: 25 - DNA, 25 bp, Artificial Sequence
  Description of Artificial Sequence Synthetic primer
tgatgagaaggaggctgaattatac

SEQ ID NO: 26 - DNA, 25 bp, Artificial Sequence
  Description of Artificial Sequence Synthetic primer
aaagtcgcacctgtaataacttgac

SEQ ID NO: 27 - DNA, 25 bp, Artificial Sequence
  Description of Artificial Sequence Synthetic primer
tatatgagatggaaggaagttctcg

SEQ ID NO: 28 - DNA, 25 bp, Artificial Sequence
  Description of Artificial Sequence Synthetic primer
atgatgtcaaagacaaggagttttc

SEQ ID NO: 29 - DNA, 45 bp, Artificial Sequence
  Description of Artificial Sequence Synthetic primer
taatatgaattaataaacacctgtccattttagaaaaacgctatg

SEQ ID NO: 30 - DNA, 45 bp, Artificial Sequence
  Description of Artificial Sequence Synthetic primer
tcgcattattaatttgtattcaaacgactaattaaaactatctta

SEQ ID NO: 31 - DNA, 74 bp, Artificial Sequence
  Description of Artificial Sequence Synthetic primer
ttttagaaaaacgctatggatgtccacgaggtctctgataccagtctcacagattccgtacgctgcaggtcgac

SEQ ID NO: 32 - DNA, 74 bp, Artificial Sequence
  Description of Artificial Sequence Synthetic primer
ctaattaaaactatcttacggtgtcggtctcgtagggatggctcacacttctttcatcgatgaattcgagctcg

SEQ ID NO: 33 - DNA, 25 bp, Artificial Sequence
  Description of Artificial Sequence Synthetic primer
aaagaacaatactgtacgccaaaag

SEQ ID NO: 34 - DNA, 25 bp, Artificial Sequence
  Description of Artificial Sequence Synthetic primer
atgtccgattgattgttgattagtt

SEQ ID NO: 35 - DNA, 25 bp, Artificial Sequence
  Description of Artificial Sequence Synthetic primer
gcagacagttcaaataggctagaag

SEQ ID NO: 36 - DNA, 25 bp, Artificial Sequence
  Description of Artificial Sequence Synthetic primer
ctaatgttgtagacactgatgtccg

SEQ ID NO: 37 - DNA, 45 bp, Artificial Sequence; identical to SEQ ID NO: 7
  Description of Artificial Sequence Synthetic primer

SEQ ID NO: 38 - DNA, 45 bp, Artificial Sequence; identical to SEQ ID NO: 8
  Description of Artificial Sequence Synthetic primer

SEQ ID NO: 39 - DNA, 74 bp, Artificial Sequence; identical to SEQ ID NO: 1
  Description of Artificial Sequence Synthetic primer

SEQ ID NO: 40 - DNA, 74 bp, Artificial Sequence; identical to SEQ ID NO: 2
  Description of Artificial Sequence Synthetic primer

SEQ ID NO: 41 - DNA, 25 bp, Artificial Sequence
  Description of Artificial Sequence Synthetic primer
agagaaatgtaaacaacagtttggg

SEQ ID NO: 42 - DNA, 25 bp, Artificial Sequence
  Description of Artificial Sequence Synthetic primer
acatgctgtaaagaatatggggata

SEQ ID NO: 43 - DNA, 25 bp, Artificial Sequence
  Description of Artificial Sequence Synthetic primer
tgtataaaattccctccaatacgaa

SEQ ID NO: 44 - DNA, 25 bp, Artificial Sequence
  Description of Artificial Sequence Synthetic primer
atagaggcttctttctttcaacgat

SEQ ID NO: 45 - DNA, 45 bp, Artificial Sequence
  Description of Artificial Sequence Synthetic primer
aaaagggctctggaagtaaacagagaaattcgacgatataatatg

SEQ ID NO: 46 - DNA, 45 bp, Artificial Sequence
  Description of Artificial Sequence Synthetic primer
cttctttagtgggttcaagacaacattatatatgcattggtatca

SEQ ID NO: 47 - DNA, 74 bp, Artificial Sequence
  Description of Artificial Sequence Synthetic primer
attcgacgatataatatggatgtccacgaggtctctgagatcatgccattcaagcccgtacgctgcaggtcgac

SEQ ID NO: 48 - DNA, 74 bp, Artificial Sequence
  Description of Artificial Sequence Synthetic primer
atatatgcattggtatcacggtgtcggtctcgtagaggcgatgatggcatttactatcgatgaattcgagctcg

SEQ ID NO: 49 - DNA, 20 bp, Artificial Sequence
  Description of Artificial Sequence Synthetic primer
ctgcagcgaggagccgtaat

SEQ ID NO: 50 - DNA, 22 bp, Artificial Sequence
  Description of Artificial Sequence Synthetic primer
tgattttgatgacgagcgtaat

SEQ ID NO: 51 - DNA, 20 bp, Artificial Sequence
  Description of Artificial Sequence Synthetic primer
cgtacgctgcaggtcgacgg

SEQ ID NO: 52 - DNA, 22 bp, Artificial Sequence
  Description of Artificial Sequence Synthetic primer
atcgatgaattcgagctcgttt

SEQ ID NO: 53 - DNA, 38 bp, Artificial Sequence
  Description of Artificial Sequence Synthetic primer
taatgtctggaacatttagttatgatgtgaaaacaaag

SEQ ID NO: 54 - DNA, 36 bp, Artificial Sequence
  Description of Artificial Sequence Synthetic primer
tgaaaaggcaatcaatccactgttttctttttccat

(19) United States
(12) Patent Application Publication
(10) Pub. No.: US 2008/0139673 A1
(43) Pub. Date: Jun. 12, 2008

(54) POTENTIATION OF ANTIFUNGAL COMPOUNDS

(75) Inventors: Wenqi Hu, Pointe Claire (CA); Nafsika Georgopapadakou, Vaudreuil-Dorion (CA)

Correspondence Address: KEOWN & ZUCCHERO, LLP, 500 WEST CUMMINGS PARK, SUITE 1200, WOBURN, MA, 01801, US

(73) Assignee: MethylGene Inc.

(21) Appl. No.: 11/836,364

(22) Filed: Aug. 9, 2007

Related U.S. Application Data
(60) Provisional application No. 60/822,192, filed on Aug. 11, 2006.

(51) Int. Cl. A61K 35/00; A01N 25/00; A61P 31/10; A01P 3/00; C12Q 1/68; G01N 33/569; C12Q 1/02

(52) U.S. Cl. 514/789; 435/6; 435/7.31; 435/29

(57) ABSTRACT

The invention provides methods and models for understanding how HDAC inhibitors interact with antifungal azole compounds to potentiate the activity of such compounds, using fungal strains which have selective knockouts of fungal HDAC genes. The invention further provides methods for testing antifungal agents for potential synergy with fungal HDAC inhibitors, and thus provides antifungal compound which are identified according to the methods of the invention, and methods for treatment of fungal infections using such compounds.

### RELATED APPLICATIONS

This application claims the benefit of U.S. Provisional Application Ser. No. 60/822,192, filed Aug. 11, 2006. The entire teachings of the above-referenced application is incorporated herein by reference.

### BACKGROUND OF THE INVENTION

(a) Field of the Invention

The invention relates to the development of antifungal drugs and the treatment of fungal infection. More particularly, the invention relates to methods of using fungal histone deacetylases as targets for potentiating antifungal drug activity. The invention further relates to the use of fungal mutations affecting histone deacetylase activity to identify compounds suitable as antifungal agents.

(b) Summary of the Related Art

In eukaryotic cells, nuclear DNA associates with histones to form a compact complex called chromatin. The histones constitute a family of basic proteins which are generally highly conserved across eukaryotic species. The core histones, termed H2A, H2B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, Biochem. J. 265: 23-38 (1990) teaches that histones are subject to post-translational acetylation of the ε-amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al., Science 272: 408-411 (1996), teaches that access of transcription factors to chromatin templates is enhanced by histone hyperacetylation. Taunton et al. (supra) further teach that an enrichment in under-acetylated histone H4 has been found in transcriptionally silent regions of the genome.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Yang and Grégoire, Mol. Cell. Biol. 25:2873-2884 (2005) teaches that, based on phylogenetic analyses and sequence homology to yeast Rpd3 (reduced potassium dependency 3), Hda1 and Sir2 (silent information regulator 2), HDACs are grouped into distinct classes. In humans there are 18 known HDACs, which are divided into four classes: class I (HDAC1, -2, -3 and -8; homologous to Rpd3), class II (HDAC4, -5, -6, -7, -9 and -10; related to Hda1), class III (Sirt1, -2, -3, -4, -5, -6 and -7; similar to Sir2) and class IV (HDAC11). Class I, II and IV HDACs are zinc-dependent enzymes. Class III HDACs are NAD⁺ dependent deacetylases. In Saccharomyces cerevisiae there are 10 known HDACs, which are divided into three classes: class I (Rpd3, Hos1 and Hos2), class II (Hda1 and Hos3), and class III (Sir2 and four Hst proteins (Hst1 to Hst4), homologs of Sir2).

It has been unclear what roles these individual HDAC enzymes play. Trojer et al., Nucleic Acids Research 31:3971-3981 (2003) indicate that HdaA and RpdA are major contributors to total HDAC activity of the filamentous fungus Aspergillus nidulans, with HdaA accounting for the main part of the HDAC activity.

Studies utilizing known HDAC inhibitors have established a link between acetylation and gene expression. Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., Trends Biochem. Sci. 25:121-126 (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

Numerous studies of fungal histone deacetylases have been reported. Giaver et al., Nature 418: 387-391 (2002) discloses six HDAC homologs (RPD3, HDA1, HOS1, HOS2, HOS3 and SIR2) in Saccharomyces cerevisiae which share high sequence homology to each other, and none of which are essential for yeast growth and survival. Bernstein et al., Proc. Natl. Acad. Sci. USA 97: 13708-13713 (2000) teaches that the role of each HDAC in yeast cells has been elusive. Suka et al., Cold Spring Harb. Symp. Quant. Biol. 63: 391-399 (1998) teaches that RPD3p is required for deacetylation of all lysines in the core histones H3, H4, H2A and H2B except for lysine 16 in histone H4. However, Wu et al., Mol. Cell. 7: 117-126 (2001) teaches that HDA1p specifically deacetylates histones H2B and H3. In, contrast, Wang et al., Science 298: 1412-1414 (2002) teaches that HOS2p binds to the coding region of genes primarily during gene activation, when it specifically deacetylates the lysines in H3 and H4 histone tails. Wang et al. also teaches that HOS2p is preferentially associated with genes of high activity genome wide, and that in combination with RPD3p it deacetylates the coding region of Erg 11, the molecular target of antifungal azoles. Rundlett et al., Proc. Natl. Acad. Sci. USA 93: 14503-14508 (1996) and Trojer et al., Nucleic Acids Res. 31: 3971-3981 (2003) show that the S. cerevisiae HOS2 is highly homologous with HOS2 in Candida. albicans and Aspergillus. fumigatus.

Numerous other reports have been published describing inhibitors of HDAC activity. For example, Richon et al., Proc. Natl. Acad. Sci. USA 95: 3003-3007 (1998), discloses that HDAC activity is inhibited by trichostatin A (TSA), a natural product isolated from Streptomyces hygroscopicus, which has been shown by Yoshida et al., J. Biol. Chem. 265: 17174-17179 (1990) and Yoshida et al., Exp. Cell Res. 177: 122-131 (1998) to inhibit histone deacetylase activity and arrest cell cycle progression in cells in the G1 and G2 phases, and by a synthetic compound, suberoylanilide hydroxamic acid (SAHA). Yoshida and Beppu, Exper. Cell Res. 177: 122-131 (1998) teaches that TSA causes arrest of rat fibroblasts at the G₁ and G₂ phases of the cell cycle, implicating HDAC in cell cycle regulation. Indeed, Finnin et al., Nature 401:188-193 (1999), teaches that TSA and SAHA inhibit cell growth, induce terminal differentiation, and prevent the formation of tumors in mice. Other non-limiting examples of compounds that serve as HDAC inhibitors include those of WO 01/38322 and WO 01/70675. Trojer et al., supra, teaches that the A. nidulans Hda1 enzyme is highly sensitive to the HDAC inhibitor TSA, while HosB has been shown to be highly resistant to both TSA and another HDAC inhibitor, HC toxin.

Smith and Edlind, Antimicrob. Agents Chemother. 46:3532-3539 (2002) tested the ability of known HDAC pan-inhibitors TSA, apicidin, sodium butyrate and trapoxin to enhance the sensitivity of selected fungal species to azole antifungal agents. They found that only TSA was able to enhance the sensitivity of C. albicans. However, the concentrations of TSA required were higher than those toxic to mammalian cells. TSA was not found to enhance the sensitivity of Candida glabrata. Co-pending application 60/751,703 teaches that several HDAC inhibitors potentiate antifungal azoles against C. albicans, C. glabrata and A. fumigatus. However, the precise molecular target and mechanism of synergy with azole antifungals remains unknown.

The use of, and need for, antifungal agents is widespread and ranges from the treatment of mycotic infections in animals and plants; to disinfectant formulations; to pharmaceuticals for human use. A major problem with current antifungal formulations is their toxicity to the infected host. This is particularly important in cases where many fungal infestations are advantageous secondary infections to debilitating diseases, such as AIDS, or from chemotherapy or transplants. Correspondingly, at least for antifungal agents that are to be administered to humans and other animals, the therapeutic index is preferably such that toxicity is selective to the targeted fungus without being toxic to the host.

Georgopapadakou, Curr. Opin. Microbiol. 1:547-557 (1998) teaches that serious fungal infections, caused mostly by opportunistic species such as Candida spp. and Aspergillus spp., are increasingly common in immunocompromised and other vulnerable patients. They are important causes of morbidity and mortality in hospitalized patients and in HIV, cancer and transplant patients. Cryptococcosis fungal infections are also extremely common is AIDS patients. Pneumocystis carinii is a major cause of death in HIV-infected patients in North America and Europe.

Infections by Candida are commonly treated with antifungal azoles which target lanosterol demethylase, an essential enzyme in ergosterol synthesis, the major component of the fungal membrane. Kaur et al., Antimicrob. Agents Chemother., 48:1600-1613 (2004) teaches that azoles are fungistatic and their use may be eroded by the emergence of azole-resistance, particularly in non-albicans Candida species such as C. glabrata. Further, azole treatment results in “trailing growth”, with surviving fungal cells becoming reservoirs for relapse. The major limitation of antifungal azoles is their lack of fungicidal activity, which may contribute to treatment failures common with severely compromised patients.

Vonberg and Gastmeier J. Hosp. Infect. 63:246-254 (2006) teaches that A. fumigatus is the major Aspergillus species causing invasive aspergillosis (IA), a life-threatening disease with a mortality rate of 60-90%, whose incidence has increased dramatically in the past 20 years due to the increasing numbers of immunocompromised patients. Current antifungal agents are limited in the treatment of IA by their poor in vivo efficacy and host toxicity (Latge, Clinical Microbiol. Rev., 12:310-350 (1999)).

Other fungal infections which are frequently fatal, especially in a debilitated patient, include cryptococcosis (infection by Cryptococcus neoformans) and zygomycosis (infection by zygomycetes).

Drawbacks to current antifungal agents, such as the azoles, include development of resistance, possible drug-drug interactions and possible toxic liver effects.

It would be highly desirable to be provided with new compositions and methods to treat fungal infection. It would also be highly desirable to be provided with compositions and methods for enhancing fungal sensitivity to antifungal compounds. Of particular importance, it would be highly desirable to provide such compositions and methods which are selectively toxic to the pathological fungus without being toxic to the host. There is, therefore, a need to better understand the precise molecular targets and mechanism of synergy of fungal HDACs with antifungal compounds.

### BRIEF SUMMARY OF THE INVENTION

The invention provides new insights into the precise targets and mechanisms of synergy of fungal HDACs with antifungal compounds.

In a first aspect, the invention provides models for understanding how HDAC inhibitors potentiate the activity of antifungal agents. According to this aspect, the invention provides fungal strains which have selective knockouts of fungal HDAC genes. These strains and their use demonstrate the importance of fungal HDAC HOS2 and its gene product, Hos2, in this regard.

In a second aspect, the invention provides a molecular target for the action of HDAC inhibitors in potentiating the activity of antifungal agents. According to this aspect, the invention provides histone deacetylase fungal mutant strains which have selective mutations of fungal HDAC genes. These strains and their use demonstrate the importance of fungal HDAC HOS2 in this regard. According to this aspect, the invention provides a HDAC deletion mutant fungal strain. According to this aspect, the invention provides a HOS2 deletion mutant strain. According to this aspect, the invention provides HOS2 and/or Hos2 as a molecular target for the action of HDAC inhibitors in potentiating the activity of antifungal agents.

In a third aspect, the invention provides methods for using a HDAC mutant fungal strain in an assay to screen a compound for potential antifungal activity and/or to screen for a compound with the ability to potentiate an antifungal agent. According to this aspect, the invention provides fungal strains which have selective knockouts of fungal HDAC genes. These strains and their use demonstrate the importance of fungal HDAC HOS2 and its gene product in this regard. According to this aspect, the invention provides for a whole-cell assay to screen a compound for potential antifungal activity and/or to screen for a compound with the ability to potentiate an antifungal agent. According to this aspect, the invention also provides for a high throughput screening assay.

In a fourth aspect, the invention provides methods for using a fungal HDAC gene and/or gene product in an assay to screen a compound for potential antifungal activity and/or to screen for a compound with the ability to potentiate an antifungal agent. According to this aspect, the invention provides a fungal HDAC gene and/or gene product to screen a compound for potential antifungal activity and/or to screen for a compound with the ability to potentiate an antifungal agent. The genes and/or gene products demonstrate the importance of fungal HDAC HOS2 (the gene) and Hos2 (the gene product) in this regard. According to this aspect, the invention also provides for a high throughput screening assay.

In a fifth aspect, the invention provides methods for testing antifungal agents for potential synergy with fungal HDAC inhibitors. According to this aspect of the invention, test strains provided by the invention provide the ability to evaluate the relative sensitivity of the HOS2 knockout with other HDAC knockout and wild-type strains.

Thus, in a sixth aspect the invention provides an antifungal compound which is identified according to the methods of the invention. This aspect further provides for a composition comprising said antifungal compound.

In a seventh aspect, the invention provides a method for inhibiting fungal growth. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In an eighth aspect, the invention provides a method for inhibiting trailing growth of a fungus in the presence of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a ninth aspect, the invention provides a method for inhibiting resistance of a fungus to an antifungal compound. The method according to this aspect of the invention comprises inhibiting the expression of fungal HOS2 or a homolog thereof, or inhibiting the activity of a gene product thereof, and treating the fungus with the antifungal compound, the activity of which is potentiated by inhibition of expression of the HOS2 or a homolog thereof or inhibition of activity of a gene product thereof.

In a tenth aspect, the invention provides a method for inhibiting survival of a fungus in the presence of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In an eleventh aspect, the invention provides a method for reducing ability of a fungus to resist antifungal activity of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a twelfth aspect, the invention provides a method for enhancing fungal sensitivity to an antifungal compound, the activity of which is potentiated by inhibition of expression of HOS2 or a homolog thereof or inhibition of activity of a HOS2 gene product or homolog thereof. The method according to this aspect of the invention comprises inhibiting the expression of fungal HOS2 or a homolog thereof, or inhibiting activity of a gene product thereof.

In a thirteenth aspect, the invention provides a method for potentiating the antifungal activity of an antifungal compound, the activity of which is potentiated by inhibition of expression of HOS2 or a homolog thereof or inhibition of activity of a HOS2 gene product, or homolog thereof. The method according to this aspect of the invention comprises inhibiting the expression of fungal HOS2 or a homolog thereof, or inhibiting activity of a gene product thereof.

In a fourteenth aspect, the invention provides a method for therapeutically treating an organism having a fungal infection. The method according to this aspect of the invention comprises inhibiting the expression of fungal HOS2 or a fungal homolog thereof, or inhibiting the activity of a gene product thereof, in an organism having a fungal infection, and treating the organism with an antifungal compound, the activity of which is potentiated by inhibition of expression of the HOS2 or a fungal homolog thereof or inhibition of activity of a gene product thereof.

In a fifteenth aspect, the invention provides a method for preventing a fungal infection in an organism. The method according to this aspect of the invention comprises treating the organism with an antifungal compound, the activity of which is potentiated by inhibition of expression of the HOS2 or a homolog thereof or inhibition of activity of a gene product thereof, and treating the organism with a HOS2 and/or a Hos2 inhibitor compound.

In a sixteenth aspect, the invention provides an inhibitor of Hos2, or a homolog thereof, or fungal HOS2, or a homolog thereof. In a preferred embodiment of this aspect, the inhibitor of Hos2, or the homolog thereof, is a hydroxymate compound.

In a seventeenth aspect, the invention provides a method of inhibiting Hos2 comprising contacting the Hos2 with the inhibitor.

In an eighteenth aspect, the invention provides a composition comprising the inhibitor of Hos2, or a homolog thereof, or the inhibitor of fungal HOS2, or a homolog thereof and a pharmaceutically acceptable carrier.

The foregoing merely summarizes certain aspects of the invention and is not intended to be limiting in nature. These aspects and other aspects and embodiments are described more fully below.

### BRIEF DESCRIPTION OF THE DRAWINGS

FIG. 1 illustrates shows a schematic representation of the strategy used to generate a yeast deletion mutant.

FIG. 2 illustrates results of (a) successful and (b) unsuccessful implementation of the strategy shown in FIG. 1.

FIG. 3 shows that a ScRPD3 deletion mutant is not hypersensitive to ketoconazole or fluconazole.

FIG. 4 shows that a ScHOS2 deletion mutant is hypersensitive to ketoconazole.

FIG. 5 shows that a ScHOS2 deletion mutant is hypersensitive to itraconazole.

FIG. 6 shows that a ScHOS2 deletion mutant is hypersensitive to voriconazole.

FIG. 7 shows that a ScHOS2 deletion mutant is hypersensitive to fluconazole.

FIG. 8 shows that a ScHOS2 deletion mutant is hypersensitive to fenpropimorph.

FIG. 9 shows that a ScHOS2 deletion mutant is not hypersensitive to terbinafine.

FIG. 10 shows that a ScHOS2 deletion mutant is not hypersensitive to amphotericin B.

FIG. 11 shows that a ScHOS2 deletion mutant is not hypersensitive to 5-fluorocytosine.

FIG. 12 shows that a ScHOS2 deletion mutant is not hypersensitive to nikkomycin.

FIG. 13 shows that ScHOS3 and ScHda1 deletion mutants are not hypersensitive to itraconazole.

FIG. 14 shows the ability of recombinant ScHos2p to complement Δhos2 susceptibility to itraconazole.

### DETAILED DESCRIPTION OF THE PREFERRED EMBODIMENTS

The present invention relates to the development of antifungal drugs. More particularly, the invention relates to methods of using fungal histone deacetylases as targets for potentiating antifungal drug activity. The invention provides new insights into the precise targets and mechanisms of synergy of fungal HDACs with antifungal compounds. To this end, the invention provides models for understanding how HDAC inhibitors potentiate the activity of antifungal compounds, using fungal strains which have selective knockouts of fungal HDAC genes. These strains and their use demonstrate the importance of fungal HDAC HOS2 in this regard. The invention further provides methods for testing antifungal agents for potential synergy with fungal HDAC inhibitors, and thus provides antifungal compounds which are identified according to the methods of the invention.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

In a first aspect, the invention provides models for understanding how HDAC inhibitors potentiate the activity of antifungal agents. According to this aspect, the invention provides fungal strains which have selective knockouts each of the fungal HDAC genes. These strains and their use demonstrate the importance of fungal HDAC HOS2 in this regard. The fungal strains providing this model include S. cerevisiae strains with single knockouts of RPD3, HDA1, HOS1, HOS2, HOS3 or SIR2, and are preferably prepared as further described herein.

In a second aspect, the invention provides a molecular target for the action of HDAC inhibitors in potentiating the activity of antifungal agents. According to this aspect, the invention provides histone deacetylase fungal mutant strains which have selective mutations of fungal HDAC genes. These strains and their use demonstrate the importance of fungal HDAC HOS2 in this regard. According to this aspect, the invention provides a HDAC deletion mutant fungal strain. According to this aspect, the invention provides a HOS2 deletion mutant strain. According to this aspect, the invention provides HOS2 and or Hos2 as a molecular target for the action of HDAC inhibitors in potentiating the activity of antifungal agents.

In a preferred embodiment according to this aspect of the invention, the histone deacetylase fungal mutant strain has a selective mutation which inhibits or prevents expression of a fungal HDAC gene. In a preferred embodiment according to this aspect of the invention, the selective mutation provides for expression of a gene product with inhibited HDAC activity. In a preferred embodiment according to this aspect, the selective mutation provides for expression of an inactive HDAC enzyme. In a preferred embodiment according to this aspect of the invention, the histone deacetylase gene is HOS2 or a homolog thereof. In a preferred embodiment according to this aspect of the invention the gene product is Hos2, or a homolog thereof.

In a preferred embodiment according to this aspect of the invention, the fungal strain is a S. cerevisiae fungal strain. In another preferred embodiment according to this aspect of the invention, the fungal strain is a fungal strain able to infect another organism, preferably a mammal, more preferably a human. In another preferred embodiment according to this aspect of the invention, the fungal strain is a pathogenic fungal strain. In a preferred embodiment according to this aspect of the invention the pathogenic fungal strain is a mammalian pathogen, preferably a human pathogen.

In a preferred embodiment according to this aspect the fungal strain is selected from the group consisting of a S. cerevisiae fungal strain, a Candida spp. fungal strain (preferably a C. albicans fungal strain, a C. glabrata fungal strain, a C. krusei fungal strain, a C. parapsilosis fungal strain or a C. tropicalis fungal strain), an Aspergillus spp. fungal strain (preferably an A. fumigatus fungal strain, an A. niger fungal strain, and an A. flavus fungal strain), a Cryptococcus neoformans fungal strain and a Pneumocystis carinii fungal strain

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a third aspect, the invention provides methods for using a HDAC mutant fungal strain in an assay to screen a compound for potential antifungal activity and/or to screen for a compound with the ability to potentiate an antifungal agent. According to this aspect, the invention provides fungal strains which have selective knockouts of fungal HDAC genes. These strains and their use demonstrate the importance of fungal HDAC HOS2 and its gene product in this regard. According to this aspect, the invention provides for a whole-cell assay to screen a compound for potential antifungal activity and/or to screen for a compound with the ability to potentiate an antifungal agent. According to this aspect, the invention also provides for a high throughput screening assay.

In preferred embodiments according to this aspect of the invention, the method comprises screening a compound for its antifungal activity. In a preferred embodiment the method identifies a compound with potential antifungal activity, said activity potentiated by inhibition of an HDAC gene, or homolog thereof, or an HDAC gene product, or homolog thereof. In preferred embodiments according to this aspect of the invention, the methods comprise providing a HDAC mutant fungal strain, the mutation resulting in loss of function of the protein encoded thereby and contacting the HDAC mutant fungal strain with the compound, wherein sensitivity of the HDAC mutant fungal strain to the test compound identifies the compound as having potential antifungal activity, said activity potentiated by inhibition of an HDAC gene, or homolog thereof, or an HDAC gene product, or homolog thereof. In a preferred embodiment the method further comprises contacting a non-HDAC mutant fungal strain, preferably a wild-type strain with the compound, wherein sensitivity of the HDAC mutant strain compared to sensitivity of the non-HDAC mutant strain identifies the compound as having potential antifungal activity, said activity potentiated by inhibition of an HDAC gene, or homolog thereof, or an HDAC gene product, or homolog thereof.

Optionally, the steps of the method for identifying a compound having potential antifungal activity, said activity potentiated by inhibition of an HDAC gene, or homolog thereof, or an HDAC gene product, or homolog thereof, can be repeated with a second HDAC mutant fungal strain. The second HDAC mutant fungal strain differs in species or genus from the first strain and has genes homologous to the mutated gene of the first strain, the homologous genes having analogous mutations.

Sensitivity to the test compound is preferably achieved by measuring killing of the fungal strain, inhibition of growth of the fungal strain, increase of surrogate markers for death, or decrease in surrogate markers for growth of the fungal strain.

In a fourth aspect, the invention provides methods for using a fungal HDAC gene and/or gene product in an assay to screen a compound for potential antifungal activity and/or to screen for a compound with the ability to potentiate an antifungal agent. According to this aspect, the invention provides a fungal HDAC gene and/or gene product to screen a compound for potential antifungal activity and/or to screen for a compound with the ability to potentiate an antifungal agent. The genes and/or gene products demonstrate the importance of fungal HDAC HOS2 (the gene) and Hos2 (the gene product) in this regard. According to this aspect, the invention also provides for a high throughput screening assay.

In preferred embodiments according to this aspect of the invention, the method comprises screening a compound for its antifungal activity and/or screening for a compound with the ability to potentiate an antifungal agent. In a preferred embodiment the method identifies a compound that inhibits a fungal HDAC gene, or homolog thereof, or a fungal HDAC gene product, or homolog thereof. In preferred embodiments according to this aspect of the invention, the methods comprise providing a fungal HDAC protein or homolog thereof, or active fragment thereof and contacting the protein or homolog thereof or active fragment thereof with the compound, wherein inhibition of HDAC activity of the protein or homolog thereof or active fragment thereof identifies the compound as having potential antifungal activity or the ability to potentiate the antifungal activity of an antifungal agent. In a preferred embodiment the fungal HDAC protein is HOS2 or a homolog thereof, or an active fragment thereof. In a preferred embodiment the method further comprises contacting a non-HDAC mutant fungal strain, preferably a wild-type strain with the compound, wherein sensitivity of the non-HDAC mutant strain identifies the compound as having potential antifungal activity. In a preferred embodiment, the method optionally comprises contacting a non-HDAC mutant fungal strain, preferably a wild-type strain with the compound and an antifungal agent, wherein sensitivity of the strain in the presence of the compound and the antifungal agent compared to the sensitivity of the strain in the presence of the antifungal agent only identifies the compound as a compound with the ability to potentiate the antifungal activity of the antifungal agent.

Sensitivity to the test compound is preferably achieved by measuring killing of the fungal strain, inhibition of the growth of the fungal strain, increase of surrogate markers for death or growth inhibition of the fungal strain, or decrease in surrogate markers for growth of the fungal strain.

Optionally, the steps of the methods for using a fungal HDAC gene and/or gene product in an assay to screen a compound for potential antifungal activity and/or to screen for a compound with the ability to potentiate an antifungal agent can be repeated with an HDAC enzyme from a second fungal strain. The second fungal strain differs in species or genus from the first strain and has enzymes homologous to the enzymes of the first strain.

In a fifth aspect, the invention provides methods for testing antifungal agents for potential synergy with fungal HDAC inhibitors, especially inhibitors of HOS2 or homologs thereof, or Hos2 or homologs thereof. According to this aspect of the invention, test strains provided by the invention provide the ability to evaluate the relative sensitivity of the HOS2 knockout with other HDAC knockout and wild-type strains.

In preferred embodiments according to this aspect of the invention, the method comprises providing a HOS2 knockout fungal strain, treating the knockout mutant strain with a test compound, determining the sensitivity of the HOS2 knockout fungal strain to the test compound, providing one or more control fungal strain selected from the group consisting of a wild-type strain, an RPD3 knockout mutant strain, an HDA1 knockout mutant strain, a HOS1 knockout mutant strain, a HOS3 knockout mutant strain and a SIR2 knockout mutant strain, treating the one or more control fungal strain with the test compound, determining the sensitivity of the one or more test strain to the test strain, and comparing the sensitivity of the HOS2 knockout strain to the test compound with the sensitivity of the one or more control strain to the test compound. The test compound is considered to be an antifungal compound having synergy with a HOS2 inhibitor if the HOS2 knockout mutant strain is more sensitive to the test compound than the one or more control strain.

Sensitivity to the test compound is preferably achieved by measuring killing of the fungal strain, inhibition of the growth of the fungal strain, increase of surrogate markers for death or growth inhibition of the fungal strain, or decrease in surrogate markers for growth of the fungal strain.

The term “knockout mutant strain” intended to mean a fungal strain comprising a mutation resulting in loss of function of the protein encoded by a HDAC gene. Such mutations include, but are not limited to, point mutations, deletion mutations, insertion mutations and inversions. Such mutations can be in coding or non-coding regions as long as they result in the loss of function of a gene product encoded by a HDAC gene or homolog thereof.

Thus, in a sixth aspect the invention provides an antifungal compound, which is identified according to the methods of the invention. This aspect further provides for a composition comprising said antifungal compound.

In a seventh aspect, the invention provides a method for inhibiting fungal growth. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In an eighth aspect, the invention provides a method for inhibiting trailing growth of a fungus in the presence of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a ninth aspect, the invention provides a method for inhibiting resistance of a fungus to an antifungal compound. The method according to this aspect of the invention comprises inhibiting the expression of fungal HOS2 or a homolog thereof, or inhibiting the activity of a gene product thereof, and treating the fungus with the antifungal compound, the activity of which is potentiated by inhibition of expression of the HOS2 or a homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a tenth aspect, the invention provides a method for inhibiting survival of a fungus in the presence of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In an eleventh aspect, the invention provides a method for reducing ability of a fungus to resist antifungal activity of an antifungal compound. The method according to this aspect of the invention comprises inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a twelfth aspect, the invention provides a method for enhancing fungal sensitivity to an antifungal compound, the activity of which is potentiated by inhibition of expression of HOS2 or a homolog thereof or inhibition of activity of a HOS2 gene product or homolog thereof. The method according to this aspect of the invention comprises inhibiting the expression of fungal HOS2 or a homolog thereof, or inhibiting activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a thirteenth aspect, the invention provides a method for potentiating the antifungal activity of an antifungal compound, the activity of which is potentiated by inhibition of expression of HOS2 or a homolog thereof or inhibition of activity of a HOS2 gene product, or homolog thereof. The method according to this aspect of the invention comprises inhibiting the expression of fungal HOS2 or a homolog thereof, or inhibiting activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a fourteenth aspect, the invention provides a method for therapeutically treating an organism having a fungal infection. The method according to this aspect of the invention comprises inhibiting the expression of fungal HOS2 or a fungal homolog thereof, or inhibiting the activity of a gene product thereof, in an organism having a fungal infection, and treating the organism with an antifungal compound, the activity of which is potentiated by inhibition of expression of the HOS2 or a fungal homolog thereof or inhibition of activity of a gene product thereof.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a preferred embodiment according to this aspect, the organism is a plant or an animal, preferably a mammal and more preferably a human. In a preferred embodiment according to this aspect of the invention, inhibiting expression of fungal HOS2 or a fungal homolog thereof, or inhibiting activity of a gene product thereof comprises administering an inhibitor of HDAC to the organism in need thereof.

In a fifteenth aspect, the invention provides a method for preventing a fungal infection in an organism. The method according to this aspect of the invention comprises treating the organism with an antifungal compound, the activity of which is potentiated by inhibition of expression of the HOS2 or a homolog thereof or inhibition of activity of a gene product thereof, and profilactively treating the organism with a HOS2 and/or a Hos2 inhibitor compound.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to this aspect of the invention, the antifungal compound is a compound which inhibits ergosterol synthesis. In another preferred embodiment the antifungal compound is an antifungal azole compound. Preferred azoles include imidazoles and triazoles. More preferred azoles include, but are not limited to, ketoconazole, itroconazole, fluconazole, voriconazole, posaconazole, ravuconazole or miconazole. In another preferred embodiment, the compound is fenpropimorph.

In a sixteenth aspect, the invention provides an inhibitor of Hos2. In a preferred embodiment of this aspect, the inhibitor is a hydroxymate compound.

In a seventeenth aspect, the invention provides a method of inhibiting Hos2 comprising contacting the Hos2 with an inhibitor of Hos2. In a preferred embodiment of this aspect, the Hos2 is in a cell, and the method comprises contacting the cell with the inhibitor. In another preferred embodiment, the cell is a fungal cell which is in or on another organism (preferably a mammalian or plant, more preferably a human), and the method comprises administering an inhibitor of Hos2 to the other organism. In a preferred embodiment of this aspect of the invention, the inhibitor of Hos2 is a compound as described herein.

In a preferred embodiment according to this aspect the fungus is selected from the group consisting of S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group.

In another preferred embodiment according to this aspect of the invention, the fungus is selected from the group consisting of zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group.

In a preferred embodiment according to the invention, the HDAC inhibitor is an inhibitor of HDAC gene expression. In another preferred embodiment according to the invention the HDAC inhibitor is an inhibitor of activity of an HDAC gene product, more preferably an enzyme.

Surprisingly, a HOS2 deletion mutant showed hypersensitivity to the antifungal azoles itraconozole, ketoconazole, fluconazole and voriconazole, each of which target lanosterol 14a-demethylase (Erg11). It did not show hypersensitivity to antifungal agents that do not act on the ergosterol biosynthetic pathway, such as amphotericin B, 5-fluorocytosine and nikkomycin. Thus, in certain preferred embodiments, test compounds according to this aspect of the invention include compounds that act on the ergosterol biosynthetic pathway.

The discovery that the absence of functional HOS2 potentiates the activity of antifungal compounds further provides a method for identifying a potentiator of an antifungal compound. The method comprises administering to a fungal strain a test compound, determining whether the test compound inhibits expression of HOS2, and identifying the test compound as a potentiator of an antifungal compound if the test compound inhibits HOS2.

An antifungal compound is considered to be potentiated by inhibition of HOS2 expression or inhibition of Hos2 activity if it is identified or identifiable as a compound having synergy with a HOS2 inhibitor by the methods described above.

For the purpose of the present invention, the following terms are defined below. Any other definitions not specifically disclosed herein are as described in co-pending application 60/751,703.

Reference to “a compound of the formula (A), formula (B), etc.,” (or equivalently, “a compound of the present invention”, and the like), herein is understood to include reference to N-oxides, hydrates, solvates, pharmaceutically acceptable salts, prodrugs and complexes thereof, and racemic and scalemic mixtures, diastereomers, enantiomers and tautomers thereof and unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B-, wherein a is 0 or 1. In such instances, when a is 0 the moiety is B- and when a is 1 the moiety is A-B-. Also, a number of moietes disclosed here may exist in multiple tautomeric forms, all of which are intended to be encompassed by any given tautomeric structure.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “aliphatic” is intended to mean both saturated and unsaturated, straight chain or branched aliphatic hydrocarbons. As will be appreciated by one of ordinary skill in the art, “aliphatic” is intended herein to include, but is not limited to, alkyl, alkenyl or alkynyl moieties.

The term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butenylene.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Preferred azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

The term “carbocycle” as employed herein is intended to mean a cycloalkyl or aryl moiety. The term “carbocycle” also includes a cycloalkenyl moiety having at least one carbon-carbon double bond.

The term “cycloalkyl” is intended to mean a saturated or unsaturated mono-, bi-, tri- or poly-cyclic hydrocarbon group having about 3 to 15 carbons, preferably having 3 to 12 carbons, preferably 3 to 8 carbons, more preferably 3 to 6 carbons, and more preferably still 5 or 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a moiety selected from the group consisting of O, S, N,N-alkyl, —S(O)—, —S(O)₂—, —S(O)₂NH—, or —NHS(O)₂—.

The term “aryl” is intended to mean a mono-, bi-, tri- or polycyclic aromatic moiety, preferably a C₆-C₁₄ aromatic moiety, preferably comprising one to three aromatic rings. Preferably, the aryl group is a C₆-C₁₀aryl group, more preferably a C₆aryl group. Preferred aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In certain preferred embodiments, the heterocyclic group is a bridged heterocyclic group (for example, a bicyclic moiety with a methylene, ethylene or propylene bridge). In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 18 ring atoms, preferably 5 to 14 ring atoms, more preferably 5, 6, 9, or 10 ring atoms; preferably having 6, 10, or 14 pi electrons shared in a cyclic array; and having, in addition to carbon atoms, between one or more heteroatoms selected from the group consisting of N, O, and S. The term “heteroaryl” is also intended to encompass the N-oxide derivative (or N-oxide derivatives, if the heteroaryl group contains more than one nitrogen such that more than one N-oxide derivative may be formed) of a nitrogen-containing heteroaryl group. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, isoxazolyl, benzo[b]thienyl, naphtha[2,3-b]thianthrenyl, zanthenyl, quinolyl, benzothiazolyl, benzimidazolyl, beta-carbolinyl and perimidinyl. Illustrative examples of N-oxide derivatives of heteroaryl groups include, but are not limited to, pyridyl N-oxide, pyrazinyl N-opxide, pyrimidinyl N-oxide, pyridazinyl N-oxide, triazinyl N-oxide, isoquinolyl N-oxide and quinolyl N-oxide.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

A heteroalicyclic group refers specifically to a non-aromatic heterocyclyl radical. A heteroalicyclic may contain unsaturation, but is not aromatic.

A heterocyclylalkyl group refers to a residue in which a heterocyclyl is attached to a parent structure via one of an alkylene, alkylidene, or alkylidyne radical. Examples include (4-methylpiperazin-1-yl) methyl, (morpholin-4-yl) methyl, (pyridine-4-yl) methyl,2-(oxazolin-2-yl)ethyl,4-(4-methylpiperazin-1-yl)-2-butenyl, and the like. If a heterocyclylalkyl is described as “optionally substituted” it is meant that both the heterocyclyl and the corresponding alkylene, alkylidene, or alkylidyne radical portion of a heterocyclylalkyl group may be optionally substituted. A “lower heterocyclylalkyl” refers to a heterocyclylalkyl where the “alkyl” portion of the group has one to six carbons.

A heteroalicyclylalkyl group refers specifically to a heterocyclylalkyl where the heterocyclyl portion of the group is non-aromatic.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolonyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, dibenzofuryl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as fuor[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

A “halohydrocarbyl” as employed herein is a hydrocarbyl moiety, in which from one to all hydrogens have been replaced with an independently selected halo.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, hydroxy, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkylamino, C₁-C₈alkoxycarbonyl, aryloxycarbonyl, C₂-C₈acyl, —C(O)—N(R³⁰)-alkyl-cycloalkyl, —C(O)—N(R³⁰)-alkyl-heterocyclyl, —C(O)—N(R³⁰)-alkyl-aryl, —C(O)—N(R³⁰)-alkyl-heteroaryl, —C(O)—cycloalkyl, —C(O)-heterocyclyl, —C(O)-aryl, —C(O)-heteroaryl, C₂-C₈acylamino, C₁-C₈alkylthio, arylalkylthio, arylthio, C₁-C₈alkylsulfinyl, arylalkylsulfonyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, heterocyclyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, heterocyclyl-C₀-C₈alkyl-NH-carbonyl, cycloalkyl-S(O)₂—, heterocyclyl-S(O)₂—, aryl-S(O)₂—, heteroaryl-S(O)₂—, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, heterocyclyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or a protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹), wherein said heterocyclyl may also be bridged (forming a bicyclic moiety with a methylene, ethylene or propylene bridge); wherein
   X³⁰ is selected from the group consisting of H, C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O-C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and
   Y³¹ is selected from the group consisting of a direct bond, —O—, —N(R³⁰)—, —C(O)—, —C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—, —C(O)—N(R³¹)—, —N(R³⁰)—C(S)—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a group, such as a hydrocarbyl, heteroalkyl, heterocyclic and/or aryl group is unsubstituted.

In other preferred embodiments, a group, such as a hydrocarbyl, heteroalkyl, heterocyclic and/or aryl group is substituted with from 1 to 4 (preferably from one to three, and more preferably one or two) independently selected substituents.

Preferred substituents on alkyl groups include, but are not limited to, hydroxyl, halogen (e.g., a single halogen substituent or multiple halo substituents; in the latter case, groups such as —CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, S(═O)₂R^{e}, P(═O)₂R^{e}, S(═O)₂OR^{e}, —P(═O)₂OR^{e}, —NR^{b}R^{c}, —NR^{b}S(═O)₂R^{e}, —NR^{b}P(═O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{e}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{e}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cylic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on carbon include those groups recited as preferred alkyl substituents. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, di-alkyl-amino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

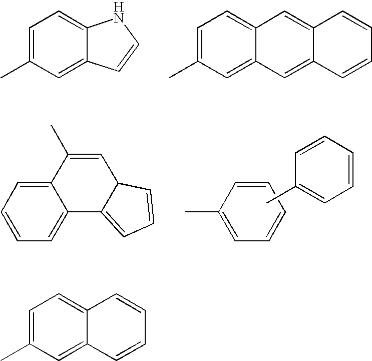

A saturated or unsaturated three- to eight-membered carbocyclic ring is preferably a four- to seven-membered, more preferably five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated three- to eight-membered heterocyclic ring contains at least one heteroatom selected from oxygen, nitrogen, and sulfur atoms. The saturated or unsaturated three- to eight-membered heterocyclic ring preferably contains one or two heteroatoms with the remaining ring-constituting atoms being carbon atoms. The saturated or unsaturated three- to eight-membered heterocyclic ring is preferably a saturated or unsaturated four- to seven-membered heterocyclic ring, more preferably a saturated or unsaturated five- or six-membered heterocyclic ring. Examples of saturated or unsaturated three- to eight-membered heterocyclic groups include thienyl, pyridyl, 1,2,3-triazolyl, imidazolyl, isoxazolyl, pyrazolyl, piperazinyl, piperazino, piperidyl, piperidino, morpholinyl, morpholino, homopiperazinyl, homopiperazino, thiomorpholinyl, thiomorpholino, tetrahydropyrrolyl, and azepanyl.

A saturated or unsaturated carboxylic and heterocyclic group may condense with another saturated or heterocyclic group to form a bicyclic group, preferably a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

In one aspect, the invention provides compounds which are inhibitors of Hos2, active fragments thereof, and homologs thereof, having the formula (A):

Cy¹-L¹—Ar¹—Y¹—C(O)—NH-Z¹ (A)

and N-oxides, hydrates, solvates, pharmaceutically acceptable salts, prodrugs or complexes thereof, and racemic and scalemic mixtures, diastereomers and enantiomers thereof, wherein Cy¹ is —H, cycloalkyl, aryl, heteroaryl, or heterocyclyl, any of which may be optionally substituted;
L¹ is —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—;
Ar¹ is arylene, wherein said arylene optionally may be additionally substituted and optionally may be fused to an aryl or heteroaryl ring, or to a saturated or partially unsaturated cycloalkyl or heterocyclic ring, any of which may be optionally substituted;
Y¹ is a chemical bond or a straight- or branched-chain saturated alkylene, wherein said alkylene may be optionally substituted; and
Z¹ is selected from the group consisting of anilinyl, pyridyl, thiadiazolyl, each of which is optionally substituted, and —O-M, M being H or a pharmaceutically acceptable cation.

In a preferred embodiment of compounds of Formula (A), when L¹ is —C(O)NH—, Y¹ is —(CH2)n —, n being 1, 2 or 3, and Z¹ is —O-M, then Cy¹ is not aminophenyl, dimethylaminophenyl, or hydroxyphenyl.

In another preferred embodiment of the compounds of Formula (A), when L¹ is —C(O)NH—, and Z¹ is pyridyl, then Cy¹ is not substituted indolinyl.

In certain preferred embodiments, Cy¹ is C₆-C₁₄ aryl, more preferably C₆-C₁₀ aryl, and most preferably phenyl or naphthyl, any of which may be optionally substituted. In certain other preferred embodiments, Cy¹ is heteroaryl. In some preferred embodiments, the heteroaryl group is selected from the group consisting of thienyl, benzothienyl, furyl, benzofuryl, quinolyl, isoquinolyl, and thiazolyl, any of which may be optionally substituted. In certain particularly preferred embodiments, Cy¹ is selected from the group consisting of phenyl, naphthyl, thienyl, benzothienyl, and quinolyl, any of which may be optionally substituted. In certain other preferred embodiments, Cy¹ is phenyl, pyridine or indole, more preferably phenyl or indole. In certain preferred embodiments, Cy¹ is substituted with one or more substituents selected from the group consisting of trihaloalkyl (preferably trifluoroalkyl), halogen, CN, amidine, sulfone, alkylsulfone, imidate and alkylimidate. In certain preferred embodiments, Cy¹ is phenyl substituted with one or more substituents selected from the group consisting of trihaloalkyl (preferably trifluoroalkyl), halogen, CN, amidine, sulfone, alkylsulfone, imidate and alkylimidate, preferably selected from the group consisting of trihaloalkyl (preferably trifluoroalkyl) and halogen.

In a preferred embodiment, L¹ is —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—. Preferably, m is 0, 1, or 2, more preferably 0 or 1.

Preferably, Ar¹ is C₆-C₁₄ arylene, more preferably C₆-C₁₀ arylene, any of which may be additionally substituted. In certain preferred embodiments, Ar¹ is phenylene, preferably 4-phenylene. In some preferred embodiments, the phenylene is fused to an aryl or heteroaryl ring, or to a saturated or partially unsaturated cycloalkyl or heterocyclic ring, any of which groups also may be optionally substituted.

In a preferred embodiment, Y¹ is a chemical bond or is a straight- or branched-chain alkylene, which may be optionally substituted. In some preferred embodiments, Y¹ is a chemical bond, and the group —C(O)NH-Z is directly attached to Ar¹. In some other preferred embodiments, Y¹ is alkylene, preferably saturated alkylene. Preferably, the saturated alkylene is C₁-C₈ alkylene, more preferably C₁-C₆ alkylene, still more preferably C₁-C₃ alkylene, and yet still more preferably C₁-C₂ alkylene, any of which may be optionally substituted. In some particularly preferred embodiments, Y¹ is methylene.

Substituted alkyl, aryl, heterocyclyl, and heteroaryl groups have one or more, preferably between one and about three, more preferably one or two substituents, which are preferably selected from the group consisting of C₁-C₆ alkyl, preferably C₁-C₄ alkyl; halo, preferably Cl, Br, or F; haloalkyl, preferably (halo)₁₋₅(C₁-C₆)alkyl, more preferably (halo)₁₋₅(C₁-C₃)alkyl, and most preferably CF₃; C₁-C₆ alkoxy, preferably methoxy, ethoxy, or benzyloxy; C₆-C₁₀ aryloxy, preferably phenoxy; C₁-C₆ alkoxycarbonyl, preferably C₁-C₃ alkoxycarbonyl, most preferably carbomethoxy or carboethoxy; C₆-C₁₀ aryl, preferably phenyl; (C₆-C₁₀)ar(C₁-C₆)alkyl, preferably (C₆-C₁₀)ar(C₁-C₃)alkyl, more preferably benzyl, naphthylmethyl or phenethyl; hydroxy(C₁-C₆)alkyl, preferably hydroxy(C₁-C₃)alkyl, more preferably hydroxymethyl; amino(C₁-C₆)alkyl, preferably amino(C₁-C₃)alkyl, more preferably aminomethyl; (C₁-C₆)alkylamino, preferably methylamino, ethylamino, or propylamino; di-(C₁-C₆)alkylamino, preferably dimethylamino or diethylamino; (C₁-C₆)alkylcarbamoyl, preferably methylcarbamoyl, dimethylcarbamoyl, or benzylcarbamoyl; (C₆-C₁₀)arylcarbamoyl, preferably phenylcarbamoyl; (C₁-C₆)alkaneacylamino, preferably acetylamino; (C₆-C₁₀)areneacylamino, preferably benzoylamino; (C₁-C₆)alkanesulfonyl, preferably methanesulfonyl; (C₁-C₆)alkanesulfonamido, preferably methanesulfonamido; (C₆-C₁₀)arenesulfonyl, preferably benzenesulfonyl or toluenesulfonyl; (C₆-C₁₀)arenesulfonamido, preferably benzenesulfonyl or toluenesulfonyl; (C₆-C₁₀)ar(C₁-C₆)alkylsulfonamido, preferably benzylsulfonamido; C₁-C₆ alkylcarbonyl, preferably C₁-C₃ alkylcarbonyl, more preferably acetyl; (C₁-C₆)acyloxy, preferably acetoxy; cyano; amino; carboxy; hydroxy; ureido; and nitro. One or more carbon atoms of an alkyl, cycloalkyl, or heterocyclyl group may also be optionally substituted with an oxo group.

In some particularly preferred embodiments, Cy1 is a phenyl, naphthyl, thienyl, benzothienyl, or quinolyl moiety which is unsubstituted or is substituted by one or two substituents independently selected from the group consisting of C₁-C₄ alkyl, C₁-C₄ haloalkyl, C₆-C₁₀ aryl, (C₆-C₁₀)ar(C₁-C₆)alkyl, halo, nitro, hydroxy, C₁-C₆ alkoxy, C₁-C₆ alkoxycarbonyl, carboxy, and amino.

In another embodiment, the invention provides compounds which are inhibitors of Hos2, active fragments thereof, and homologs thereof, represented by formula (B):

Cy²-L²—Ar²—Y²—C(O)NH-Z² (B)

and N-oxides, hydrates, solvates, pharmaceutically acceptable salts, prodrugs or complexes thereof, and racemic and scalemic mixtures, diastereomers and enantiomers thereof, wherein Cy² is H, cycloalkyl, aryl, heteroaryl, or heterocyclyl, any of which may be optionally substituted, provided that Cy² is not a (spirocycloalkyl)heterocyclyl;
L² is C₁-C₆ saturated alkylene or C₂-C₆ alkenylene, wherein the alkylene or alkenylene optionally may be substituted, and wherein one or two of the carbon atoms of the alkylene is optionally replaced by a heteroatomic moiety independently selected from the group consisting of 0; NR′,
R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z² is selected from the group consisting of anilinyl, pyridyl, thiadiazolyl, each of which is optionally substituted, and —O-M, M being H or a pharmaceutically acceptable cation.

In a preferred embodiment of the compounds of Formula (B), when the carbon atom to which Cy² is attached is oxo substituted, then Cy² and Z² are not both pyridyl.

Preferred substituents of Cy², Ar², and Z² according to this aspect of the invention are as defined above for Formula (A). Preferred substituents of Y² are as defined above for Y¹. In some preferred embodiments, L² is saturated C₁-C₈ alkylene, more preferably C₁-C₆ alkylene, still more preferably C₁-C₄ alkylene, any of which groups may be optionally substituted. In some other preferred embodiments, L² is C₂-C₈ alkenylene, more preferably C₂-C₆ alkenylene, and still more preferably C₂-C₄ alkenylene, any of which groups may be optionally substituted. The alkylene or alkenylene group may be substituted at one or more carbon positions with a substituent preferably selected from the list of preferred substituents recited above. More preferably, L² is substituted at one or two positions with a substituent independently selected from the group consisting of C₁-C₆ alkyl, C₆-C₁₀ aryl, amino, oxo, hydroxy, C₁-C₄ alkoxy, and C₆-C₁₀ aryloxy. In some particularly preferred embodiments, the alkylene or alkenylene group is substituted with one or two oxo or hydroxy groups.

In some preferred embodiments, L² is C₁-C₆ saturated alkylene, wherein on of the carbon atoms of the saturated alkylene is replaced by a heteroatom moiety selected from the group consisting of O; NR′, R′ being alkyl, acyl, or hydrogen; S; S(O); or S(O)₂. Preferably, the carbon atom adjacent to Cy2 is replaced by a heteroatom moiety. In some particularly preferred embodiments, L² is selected from the group consisting of —S—(CH₂)₂—, —S(O)—(CH₂)₂—, —S(O)₂—(CH₂)₂—, —S—(CH₂)₃—, —S(O)—(CH₂)₃—, and —S(O)₂—(CH₂)₃—.

In another preferred embodiment, the invention provides compounds which are inhibitors of Hos2, active fragments thereof, and homologs thereof, represented by formula (C):

Cy³-L³—Ar³-Y³—C(O)NH-Z³ (C)

and N-oxides, hydrates, solvates, pharmaceutically acceptable salts, prodrugs or complexes thereof, and racemic and scalemic mixtures, diastereomers and enantiomers thereof, wherein Cy³ is —H, cycloalkyl, aryl, heteroaryl, or heterocyclyl, any of which may be optionally substituted, provided that Cy³ is not a (spirocycloalkyl)heterocyclyl;
L³ is selected from the group consisting of
(a) —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—; and
(b) C₁-C₆ alkylene or C₂-C₆ alkenylene, wherein the alkylene or alkenylene optionally may be substituted, and wherein one of the carbon atoms of the alkylene optionally may be replaced by O; NR′, R′ being alkyl, acyl, or hydrogen; S; S(O); or S(O)₂;
Ar³ is arylene, wherein said arylene optionally may be additionally substituted and optionally may be fused to an aryl or heteroaryl ring, or to a saturated or partially unsaturated cycloalkyl or heterocyclic ring, any of which may be optionally substituted;
Y³ is C₂ alkenylene or C₂ alkynylene, wherein one or both carbon atoms of the alkenylene optionally may be substituted with alkyl, aryl, alkaryl, or aralkyl; and
Z³ is selected from the group consisting of anilinyl, pyridyl, thiadiazolyl, each of which is optionally substituted, and —O-M, M being H or a pharmaceutically acceptable cation.

In a preferred embodiment of the compounds of Formula (C), when Cy³ is unsubstituted phenyl, Ar³ is not phenyl wherein L³ and Y³ are oriented ortho or meta to each other.

Preferred substituents of Cy³, Ar³, and Z³ according to this aspect of the invention are as defined above for Formula (A).

In a preferred embodiment of one aspect of the invention, the invention provides compounds, preferably hydroxamte-based compounds as described in U.S. Ser. No. 10/880,444, which is incorporated herein by reference in its entirety, that are useful for inhibiting Hos2.

In a preferred embodiment of one aspect, the invention comprises hydroxamate-based compounds, preferably compounds of formula (D), that are useful for inhibiting Hos2, active fragments thereof, and homologs thereof:

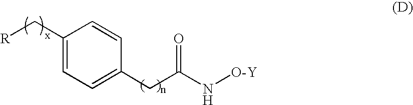

and N-oxides, hydrates, solvates, pharmaceutically acceptable salts, prodrugs and complexes thereof, and racemic and scalemic mixtures, diastereomers and enantiomers thereof, wherein R is alkyl, cycloalkyl, aryl, heteroaryl or heterocyclyl, preferably cycloalkyl, aryl, heteroaryl or heterocyclyl, any of which maybe optionally substituted;
x is an integer from 0 to 5, wherein the chain of length x is optionally substituted and wherein one or two carbon atoms of the chain of length x is optionally replaced with a heteroatom;
n is an integer from 0 to 2; and
Y is selected from the group consisting of H and a heterocyclic group; and
the phenylene is optionally substituted.

In a preferred embodiment of compounds of Formula (D), when x is 4, n is not 2, and when x is 3, n is not 3.

In a preferred embodiment of the compounds according to the present invention, R optionally has one or more, preferably between one and about three, more preferably one or two substituents, which are preferably selected from the group consisting of C₁-C₆ alkyl, preferably C₁-C₄ alkyl; halo, preferably Cl, Br, or F; haloalkyl, preferably (halo)₁₋₅(C₁-C₆)alkyl, more preferably (halo)₁₋₅(C₁-C₃)alkyl, and most preferably CF₃; C₁-C₆ alkoxy, preferably methoxy, ethoxy, or benzyloxy; C₆-C₁₀ aryloxy, preferably phenoxy; C₁-C₆ alkoxycarbonyl, preferably C₁-C₃ alkoxycarbonyl, most preferably carbomethoxy or carboethoxy; C₆-C₁₀ aryl, preferably phenyl; (C₆-C₁₀)ar(C₁-C₆)alkyl, preferably (C₆-C₁₀)ar(C₁-C₃)alkyl, more preferably benzyl, naphthylmethyl or phenethyl; hydroxy(C₁-C₆)alkyl, preferably hydroxy(C₁-C₃)alkyl, more preferably hydroxymethyl; amino(C₁-C₆)alkyl, preferably amino(C₁-C₃)alkyl, more preferably aminomethyl; (C₁-C₆)alkylamino, preferably methylamino, ethylamino, or propylamino; di-(C₁-C₆)alkylamino, preferably dimethylamino or diethylamino; (C₁-C₆)alkylcarbamoyl, preferably methylcarbamoyl, dimethylcarbamoyl, or benzylcarbamoyl; (C₆-C₁₀)arylcarbamoyl, preferably phenylcarbamoyl; (C₁-C₆)alkaneacylamino, preferably acetylamino; (C₆-C₁₀)areneacylamino, preferably benzoylamino; (C₁-C₆)alkanesulfonyl, preferably methanesulfonyl; (C₁-C₆)alkanesulfonamido, preferably methanesulfonamido; (C₆-C₁₀)arenesulfonyl, preferably benzenesulfonyl or toluenesulfonyl; (C₆-C₁₀)arenesulfonamido, preferably benzenesulfonyl or toluenesulfonyl; (C₆-C₁₀)ar(C₁-C₆)alkylsulfonamido, preferably benzylsulfonamido; C₁-C₆ alkylcarbonyl, preferably C₁-C₃ alkylcarbonyl, more preferably acetyl; (C₁-C₆)acyloxy, preferably acetoxy; cyano; amino; carboxy; hydroxy; ureido; nitro and oxo.

In another preferred embodiment of the compounds according to the present invention, R is unsubstituted or is substituted by one or two substituents independently selected from the group consisting of C₁-C₄ alkyl, C₁-C₄ haloalkyl, C₆-C₁₀ aryl, (C₆-C₁₀)ar(C₁-C₆)alkyl, halo, nitro, hydroxy, C₁-C₆ alkoxy, C₁-C₆ alkoxycarbonyl, carboxy, and amino.

In a preferred embodiment of the compounds according to the present invention, R is phenyl, pyridine or indole, more preferably phenyl or indole, more preferably phenyl.

In a preferred embodiment of the compounds according to the present invention, R is substituted with one or more substituents independently selected from the group consisting of alkyl, alkenyl, alkynyl, trihaloalkyl, halogen, CN, amidine, alkylamidine, sulfone, alkylsulfone, imidate and alkylimidate.

In a preferred embodiment of the compounds according to the present invention, R is phenyl or indole, substituted with one or more substituents independently selected from the group consisting of alkyl, alkenyl, alkynyl, trihaloalkyl, halogen, CN, amidine, alkylamidine, sulfone, alkylsulfone, imidate and alkylimidate, more preferably one or more substituents independently selected from the group consisting of alkyl, alkenyl, alkynyl, trihaloalkyl and halogen.

In a preferred embodiment of the compounds according to the present invention, x is an integer from 2 to 4, more preferably 3 to 4.

In a preferred embodiment of the compounds according to the present invention, n is an integer from 1 to 2, more preferably 1.

In a preferred embodiment of the compounds according to the present invention, Y is H.

In a preferred embodiment of the compounds according to the present invention, one carbon atom of the chain of length x is replaced with a heteroatom, preferably S.

Throughout the specification preferred embodiments of one or more chemical substituents are identified. Also preferred are combinations of preferred embodiments.

In a preferred embodiment of the compounds according to the present invention, the compound is selected from the group consisting of

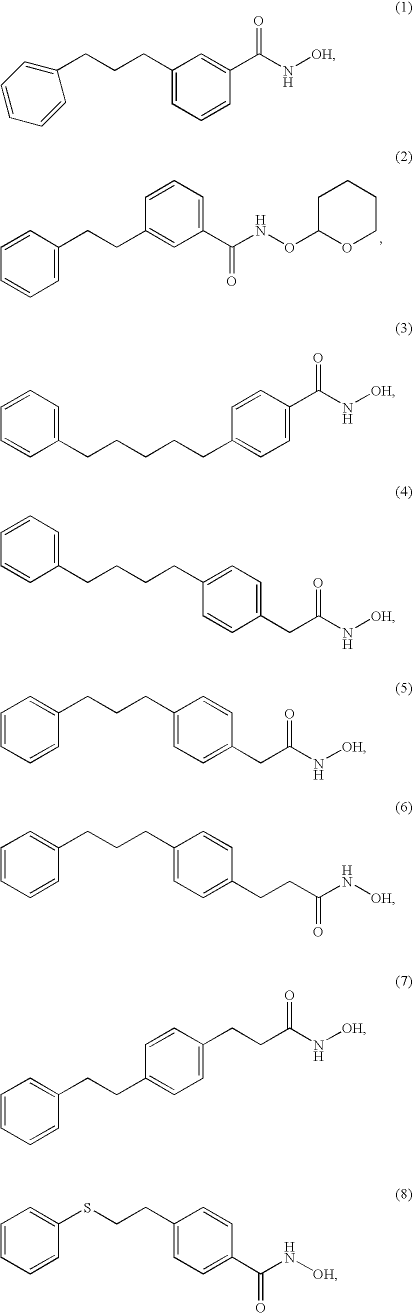

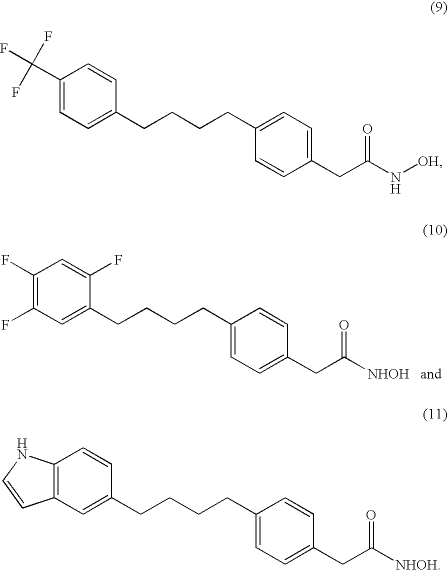

and N-oxides, hydrates, solvates, pharmaceutically acceptable salts, prodrugs or complexes thereof, and racemic and scalemic mixtures, diastereomers and enantiomers thereof.

In a preferred embodiment of the compounds according to the present invention, the compound is selected from the group consisting of

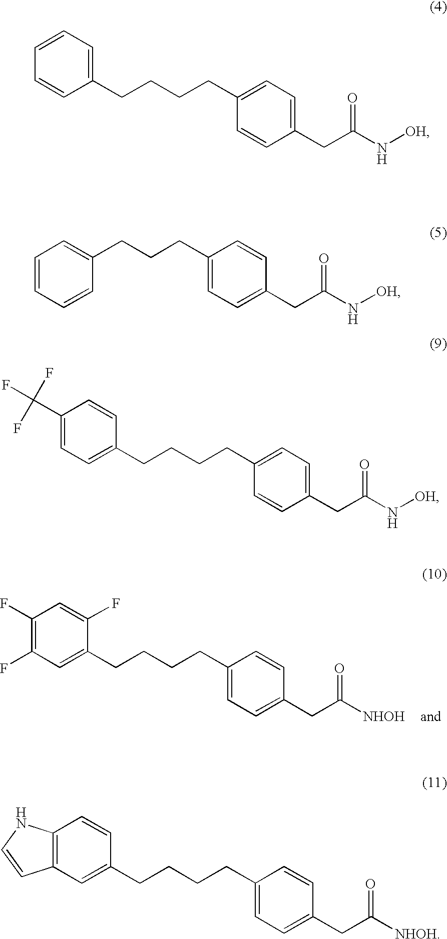

and N-oxides, hydrates, solvates, pharmaceutically acceptable salts, prodrugs or complexes thereof, and racemic and scalemic mixtures, diastereomers and enantiomers thereof.

In a preferred embodiment of the compounds according to the present invention, the compound is

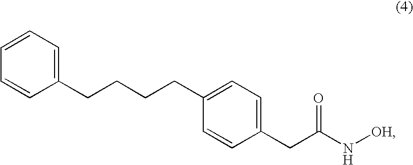

and N-oxides, hydrates, solvates, pharmaceutically acceptable salts, prodrugs or complexes thereof, and racemic and scalemic mixtures, diastereomers and enantiomers thereof.

The present invention further provides for the use of the compounds as described herein for inhibiting Hos2, active fragments thereof, and homologs thereof.

Because the compounds of Formulas (A) through (D) are useful for inhibiting Hos2, active fragments thereof, and homologs thereof, they are useful as research tools to study Hos2.

The terms RPD3, HDA1, HOS1, HOS2, HOS3 and SIR2 refer to those genes as they are known in the art, including those genes in S. cerevisiae, Candida spp. (preferably C. albicans, C. glabrata, C. krusei, C. parapsilosis and C. tropicalis), Aspergillus spp. (preferably A. fumigatus, A. terreus, A. niger and A. flavus), Cryptococcus neoformans, Pneumocystis carinii, and strains thereof of said group, and any other fungus and strains thereof (including but not limited to zygomycetes (including but not limited to Rhizopus arrhizus and Mucor spp.), emerging molds (including but not limited to Pseudallescheria boydii, Fusarium, spp. and Paecilomyces lilacinus), Coccidioides spp. (including but not limited to C. immitis), Histoplasma spp., Trichosporon spp., and dermatophytes, and strains thereof of said group), as well as any homologs thereof.

The term wild-type strain refers to a fungal strain having functional RPD3, HDA1, HOS1, HOS2, HOS3 and SIR2 gene activity.

The terms RPD3 knockout mutant strain, HDA1 knockout mutant strain, HOS1 knockout mutant strain, HOS2 knockout mutant strain, HOS3 knockout mutant strain and SIR2 knockout mutant strain refer to fungal strains in which the RPD3, HDA1, HOS1, HOS2, HOS3 or SIR2 gene, respectively, are not functional.

The term “pharmaceutically acceptable carrier” is intended to mean a non-toxic material that is compatible with a biological system in a cell, cell culture, tissue sample or body and that does not interfere with the effectiveness of the biological activity of the active ingredient(s). Thus, compositions according to the invention may contain, in addition to the inhibitor and antifungal agent, diluents, excipients, fillers, salts, buffers, stabilizers, solubilizers, and/or other materials well known in the art. Examples of the preparation of pharmaceutically acceptable formulations are described in, e.g., Remington's Pharmaceutical Sciences, 18th Edition, ed. A. Gennaro, Mack Publishing Co., Easton, Pa., 1990.

It will be understood that the characteristics of the carrier, will depend on the route of administration for a particular application.

The term “pharmaceutically acceptable salt” is intended to mean a salt that retains the desired biological activity of the above-identified compounds and exhibits minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be in the form of pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compounds of a composition of the invention are included in the pharmaceutically acceptable carrier in an amount sufficient to deliver an effective desired amount without causing serious toxic effects to an individual to which the composition is administered.

The term “homolog” is a generic term used in the art and is intended to mean a polynucleotide or polypeptide sequence possessing a high degree of sequence relatedness to a reference sequence. Such relatedness may be quantified by determining the degree of identity and/or similarity between the two sequences as determined by those of skill in the art. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide within the same species which is functionally similar.

The term “identity” is intended to mean a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

The term “similarity” is intended to mean a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two, sequences can then be determined.

For polypeptide sequences, conservative substitutions consist of substitution of one amino acid at a given position in the sequence for another amino acid of the same class (e.g., amino acids that share characteristics of hydrophobicity, charge, pK or other conformational or chemical properties, e.g., valine for leucine, arginine for lysine), or by one or more non-conservative amino acid substitutions, deletions, or insertions, located at positions of the sequence that do not alter the conformation or folding of the polypeptide to the extent that the biological activity of the polypeptide is destroyed. Examples of “conservative substitutions” include substitution of one non-polar (hydrophobic) residue such as isoleucine, valine, leucine or methionine for another; the substitution of one polar (hydrophilic) residue for another such as between arginine and lysine, between glutamine and asparagine, between threonine and serine; the substitution of one basic residue such as lysine, arginine or histidine for another; or the substitution of one acidic residue, such as aspartic acid or glutamic acid for another, or the use of a chemically derivatized residue in place of a non-derivatized residue; provided that the polypeptide displays the requisite biological activity.

Methods for comparing the identity and similarity of two or more sequences are well known in the art; for instance, the BLAST family of programs (Altschul S F et al., J Mol Biol, 215, 403-410,1990, Altschul S F et al., Nucleic Acids Res., 25:389-3402, 1997, available from the National Center for Biotechnology Information (NCBI), Bethesda, Md., USA and accessible through the home page of the NCBI at http://www.ncbi.nlm.nih.gov/).

The present invention thus also encompasses homologs of the Saccharomyces cerevisiae HOS2 gene and Hos2 protein, where the source of homologous genes and proteins can be any other fungal species, including but not limited to for example, Candida and Aspergillus, and other species and strains thereof as mentioned herein. Between fungal species, e.g., Saccharomyces and Candida, gene homologs have substantial sequence similarity, e.g., at least 50% sequence identity, at least 75% sequence identity, usually at least 90%, more usually at least 95% between nucleotide sequences.

A Hos2 protein homolog is meant a protein having at least about 35%, preferably at least about 40%, more preferably at least about 60%, even more preferably at least about 80%, more preferably at least about 90%, and more preferably still at least about 95% amino acid sequence identity to the Saccharomyces cerevisiae Hos2 protein.

The present invention also encompasses allelic variants of Hos2 polypeptides and the nucleic acids encoding them; that is, naturally-occurring alternative forms of such polypeptides and nucleic acids in which differences in amino acid or nucleotide sequence are attributable to genetic polymorphism (allelic variation among individuals within a population). Naturally and artificially occurring HOS2 mutants are also encompassed by the present invention.

Homologs and alleles of HOS2 of the invention, for example, can be identified by conventional techniques known to one skilled in the art. For example, a Candida glabrata homolog of S. cerevisiase HOS2 may be isolated and identified by making suitable probes or primers from polynucleotides encoding HOS2 and screening a suitable nucleic acid source from the desired species, for example a C. glabrata cDNA library, and selecting positive clones. Thus, an aspect of the invention are nucleic acid sequences which encode for Hos2 homolog polypeptides and which hybridize under stringent conditions to a nucleic acid molecule comprising a sequence of nucleic acid corresponding to a region of nucleic acid encoding HOS2. The term “stringent conditions” as used herein refers to parameters with which the art is familiar.

The term “antifungal agent” is intended to mean a substance capable of inhibiting or preventing the growth, viability and/or reproduction of a fungal cell. Preferable antifungal agents are those capable of preventing or treating a fungal infection in an animal or plant. A preferable antifungal agent is a broad spectrum antifungal agent. However, an antifungal agent can also be specific to one or more particular species of fungus.

The terms “histone deacetylase inhibitor” and “inhibitor of histone deacetylase” are intended to mean a compound which is capable of interacting with a histone deacetylase and inhibiting its enzymatic activity. “Inhibiting histone deacetylase enzymatic activity” means reducing the ability of a histone deacetylase to remove an acetyl group from a protein, including but not limited to a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. In some preferred embodiments of the invention, the histone deacetylase is Hos2 or a homolog thereof.

The histone deacetylase inhibitor may be any molecule that effects a reduction in the activity of a histone deacetylase. This includes proteins, peptides, antibodies and active fragments thereof, DNA molecules (including antisense), RNA molecules (including RNAi and antisense) and small molecules.

Preferably, such inhibition is specific, i.e., the histone deacetylase inhibitor reduces the ability of a histone deacetylase to remove an acetyl group from a protein, including but not limited to a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

In preferred embodiments of the present invention the histone deacetylase inhibitor inhibits one or more histone deacetylase isoforms, but less than all specific histone deacetylase isoforms. Preferred histone deacetylase isoforms include class I and class II enzymes. Specific HDACs include without limitation Rpd3, Hos1, Hos2, Hda1, Hos3, Sir2 and the Hst proteins, and homologs thereof. In a preferred embodiment of the present invention, the histone deacetylase inhibitor inhibits Hos2.

The terms “histone deacetylase gene inhibitor” and “inhibitor of histone deacetylase gene expression” are intended to mean a compound which is capable of interacting with a nucleic acid encoding for a histone deacetylase, or a nucleic acid encoding for a protein with histone deacetylase activity, and inhibiting its ability to express a product with efficient histone deacetylase activity. Inhibition of expression may be at the level of transcription and/or translation. In some preferred embodiments, such reduction of expression is at least about 50%, more preferably at least about 75%, and still more preferably at least about 90%. In other preferred embodiments, expression is reduced by at least 95% and more preferably by at least 99%. In some preferred embodiments of the invention, the nucleic acid encodes for HOS2 or a homolog thereof.

The histone deacetylase gene inhibitor may be any molecule that effects a reduction in the expression of a product with efficient histone deacetylase activity. This includes proteins, peptides, antibodies and active fragments thereof, DNA molecules (including antisense), RNA molecules (including RNAi and antisense) and small molecules.

Preferably, such inhibition is specific, i.e., the histone deacetylase gene inhibitor reduces the ability to express a product with efficient histone deacetylase activity at a concentration that is lower than the concentration of the inhibitor that is required to produce another, unrelated biological effect. Preferably, the concentration of the inhibitor required for the inhibitory activity is at least 2-fold lower, more preferably at least 5-fold lower, even more preferably at least 10-fold lower, and most preferably at least 20-fold lower than the concentration required to produce an unrelated biological effect.

The term “inhibition effective amount” is meant to denote a dosage sufficient to cause inhibition of fungal HDAC activity, preferably fungal HDAC activity in a cell, which cell can be in a multicellular organism. The fungus may be infecting a plant or a mammal, preferably a human, and could therefore be located in and/or on the plant or mammal. If the fungus is in or on a multicellular organism, the method according to this aspect of the invention comprises administering to the organism a compound or composition according to the present invention. Administration may be by any route, including, without limitation, parenteral, oral, sublingual, transdermal, topical, intranasal, intratracheal, intravenous or intrarectal. In certain particularly preferred embodiments, compounds of the invention are administered intravenously in a hospital setting. In certain other preferred embodiments, administration may preferably be by the oral route.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. Further, the therapeutic effect can be inhibition of Hos2 activity or inhibition of HOS2 expression. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

The term “patient” as employed herein for the purposes of the present invention includes humans and other animals, particularly mammals, and other organisms. Thus the compounds, compositions and methods of the present invention are applicable to both human therapy and veterinary applications. In a preferred embodiment the patient is a mammal, and in a most preferred embodiment the patient is human.

The terms “treating”, “treatment”, or the like, as used herein covers the treatment of a disease-state in an animal and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment of the present invention the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

In preferred embodiments of the present invention the histone deacetylase inhibitor inhibits one or more histone deacetylase isoforms, but less than all specific histone deacetylase isoforms. Preferred histone deacetylase isoforms include genes for class I and class II enzymes. Specific HDACs genes include without limitation RPD3, HOS1, HOS2, HDA1, HOS3, SIR2 and the HST genes, and homologs thereof. In a preferred embodiment of the present invention, the histone deacetylase inhibits HOS2.

The present invention is in no way limited to purely mammalian applications and is intended to encompass for example agricultural and aquatic applications, including for example methods for treating fungal infections of mammals, fish and plants. Smith and Edlind (supra) for example showed that TSA reduced the minimum inhibitory concentration of the morpholine fenpropimorph, an agricultural fungicide whose enzyme targets in the ergosterol biosynthetic pathway follow those of allylamines and azoles.

The present invention will be more readily understood by referring to the following examples, which are given to illustrate the invention rather than to limit its scope.

### Example 1

### Generation of Yeast HDAC Deletion Strains

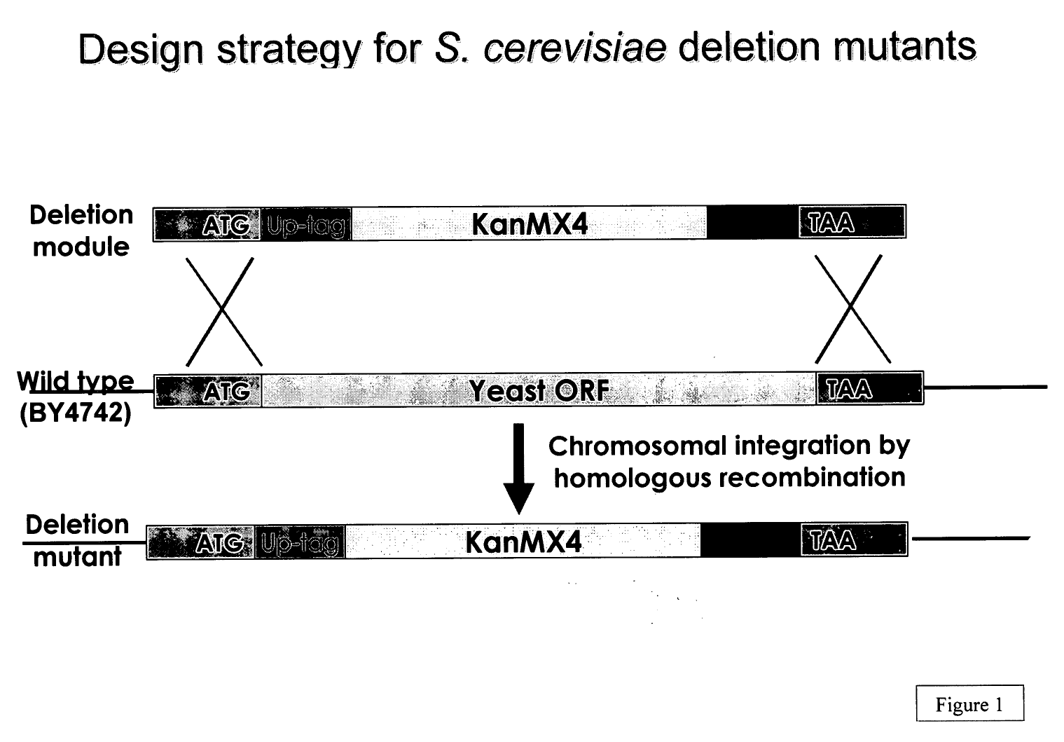

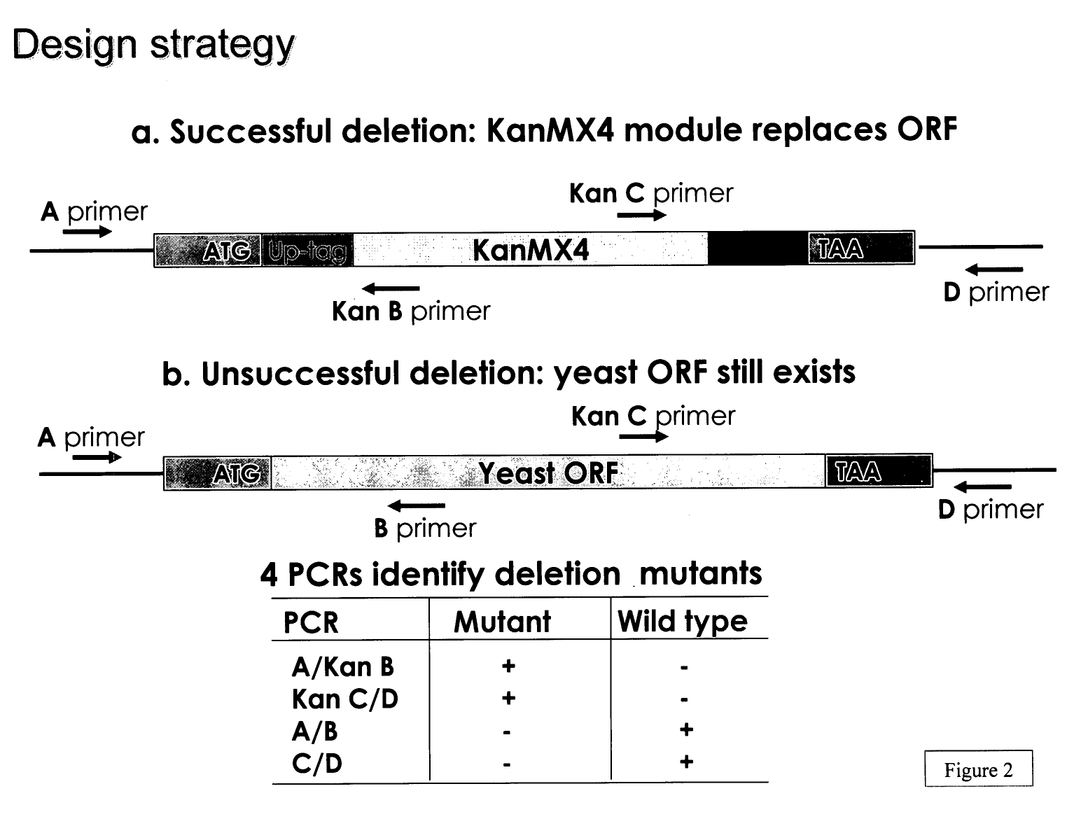

A PCR-generated deletion strategy was used to systematically replace each yeast open reading frame from its start- to stop-codon with a KanMX module and two unique 20mer molecular bar codes. (See Wach et al., Yeast 10: 1793-1808 (1994).) This strategy and potential outcomes are shown in FIGS. 1 and 2, respectively. The presence of the tags can be detected via hybridization to a high-density oligonucleotide array, enabling growth phenotypes of individual strains to be analyzed in parallel. A HOS2 deletion “cassette” was constructed using two sequential PCR reactions.

In the first amplification, 74 bp UPTAG (ATAACAACACGCAACATGGATGTCCACGAGGTCTCTTACTGGACGGCACGGTTTAT CGTACGCTGCAGGTCGAC) and 74 bp DNTAG (TAGCAAACTCTTAAACTACGGTGTCGGTCTCGTAGAACGGTTGCTAATGTTTCCGAT CGATGAATTCGAGCTCG) primers were used to amplify the KanMX gene from genomic DNA extracted from a yeast RPD3 heterozygote mutant (purchased from ATCC) which contains kanMX4 DNA whose KanMX expression confers dominant selection of geneticin (G418) to yeast. These primers consist of (5′ to 3′): 18 bp of genomic sequence that flank either the 5′ or 3′ end of the ORF (directly proximal and distal to the start and stop codons respectively), 18 and 17 bp of sequence common to all gene disruptions (U1: 5′-GATGTCCACGAGGTCTCT-3′ or D1: 5′-CGGTGTCGGTCTCGTAG-3′), a 20 bp unique sequence (the ‘molecular bar-code’ TAG) and 18 and 19 bp of sequence, respectively, homologous to the KanMX4 cassette (U2: 5′-CGTACGCTGCAGGTCGAC-3′ or D2: 5′-ATCGATGAATTCGAGCTCG-3′). PCR was performed using the following parameters: 94° C. for 3 minutes denaturation; 94° C. for 30 sec., 50° C. for 30 sec., 72° C. for 2.5 min., 35 cycle; then extension at 72° C. for 10 min.

In the second PCR reaction, two ORF specific 45-mer primers (UP_{—}45 and DOWN_{—}45) (UP-45: AGTACGTTAAAATCAGGTATCAAGTGAATAACAACACGCAACATG; DOWN_{—}45: AAAAAAAAAAACGGGAGATTAACCGAATAGCAAACTCTTAAACTA;) were used to extend the ORF specific homology to 45 bp, increasing the targeting specificity during mitotic recombination of the gene disruption cassette. PCR was performed as described above.

A standard Lithium-Acetate transformation protocol (See Gietz and Woods, Methods in Enzymology 350: 87-96 (2002)) was used to introduce the gene disruption cassette into haploid yeast cells (BY4742: MATa his3D1 leu2D0 lys2D0 ura3D0).

Other HDAC deletion mutants were prepared according to the same strategy, using the following primers for each:

| | |
|---|---|
| DNA Name | Sequence 5′-3′ |

| | |
|---|---|
| 1. RPD3-A | GATAAGATTGCGACAAAAGAGGATA |
| 2. RPD3-B | CAGTATGGAACTGACACATTTCTTG |
| 3. RPD3-C | CTAGTGTTCAGTTGAATCACACACC |
| 4. RPD3-D | GTGGGACGAGACGTTTAGATAGTAA |
| 5. RPD3-45U | GCCATACAAAACATTCGTGGCTACAACTCGATATCC |
| | GTGCAGATG |
| 6. RPD3-45D | TTTCACATTATTTATATTCGTATATACTTCCAACTC |
| | TTTTTTTCA |
| 7. RPD3-uptag | TCGATATCCGTGCAGATGGATGTCCACGAGGTCTCT |
| | ATTGCATTCGCACTTCCGATCGTACGCTGCAGGTCG |
| | AC |
| 8. RPD3-dntag | TTCCAACTCTTTTTTTCACGGTGTCGGTCTCGTAGT |
| | ATGATCGGACACCACGCAGATCGATGAATTCGAGCT |
| | CG |
| 9. HDA1-A | ATGCTTTTTCGTAGCTAACTTCTCA |
| 10. HDA1-B | TGGTCTTACGACAGCTAGAGAGTTT |
| 11. HDA1-C | ATTACTCAGGAATGATTACATCCCA |
| 12. HDA1-D | AAGTGAGTATTTGGCTCAACAGAAC |
| 13. HDA1-45U | AAAGGGAAAGTTGAGCACTGTAATACGCCGAACAGA |
| | TTAAGCATG |
| 14. HDA1-45D | ATGAAGGTTGCCGAAAAAAAATTATTAATGGCCAGT |
| | TTTTCCTCA |
| 15. HDA1-uptag | CCGAACAGATTAAGCATGGATGTCCACGAGGTCTCT |
| | AGAGTCATCCCATTACCTAGCGTACGCTGCAGGTCG |
| | AC |
| 16. HDA1-dntag | ATGGCCAGTTTTTCCTCACGGTGTCGGTCTCGTAGT |
| | AGGCTAAGAGTTGCTAACGATCGATGAATTCGAGCT |
| | CG |
| 17. HOS1-A | TGATGAGAAGGAGGCTGAATTATAC |
| 18. HOS1-B | AAAGTCGCACCTGTAATAACTTGAC |
| 19. HOS1-C | TATATGAGATGGAAGGAAGTTCTCG |
| 20. HOS1-D | ATGATGTCAAAGACAAGGAGTTTTC |
| 21. HOS1-45u | TAATATGAATTAATAAACACCTGTCCATTTTAGAAA |
| | AACGCTATG |
| 22. HOS1-45d | TCGCATTATTAATTTGTATTCAAACGACTAATTAAA |
| | ACTATCTTA |
| 23. HOS1-uptag | TTTTAGAAAAACGCTATGGATGTCCACGAGGTCTCT |
| | GATACCAGTCTCACAGATTCCGTACGCTGCAGGTCG |
| | AC |
| 24. HOS1-dntag | CTAATTAAAACTATCTTACGGTGTCGGTCTCGTAGG |
| | GATGGCTCACACTTCTTTCATCGATGAATTCGAGCT |
| | CG |
| 25. HOS2-A | AAAGAACAATACTGTACGCCAAAAG |
| 26. HOS2-B | ATGTCCGATTGATTGTTGATTAGTT |
| 27. HOS2-C | GCAGACAGTTCAAATAGGCTAGAAG |
| 28. HOS2-D | CTAATGTTGTAGACACTGATGTCCG |
| 29. HOS2-45u | AGTACGTTAAAATCAGGTATCAAGTGAATAACAACA |
| | CGCAACATG |
| 30. HOS2-45d | AAAAAAAAAAACGGGAGATTAACCGAATAGCAAACT |
| | CTTAAACTA |
| 31. HOS2-uptag | ATAACAACACGCAACATGGATGTCCACGAGGTCTCT |
| | TACTGGACGGCACGGTTTATCGTACGCTGCAGGTCG |
| | AC |
| 32. HOS2-dntag | TAGCAAACTCTTAAACTACGGTGTCGGTCTCGTAGA |
| | ACGGTTGCTAATGTTTCCGATCGATGAATTCGAGCT |
| | CG |
| 33. HOS3-A | AGAGAAATGTAAACAACAGTTTGGG |
| 34. HOS3-B | ACATGCTGTAAAGAATATGGGGATA |
| 35. HOS3-C | TGTATAAAATTCCCTCCAATACGAA |
| 36. HOS3-D | ATAGAGGCTTCTTTCTTTCAACGAT |
| 37. HOS3-45u | AAAAGGGCTCTGGAAGTAAACAGAGAAATTCGACGA |
| | TATAATATG |
| 38. HOS3-45d | CTTCTTTAGTGGGTTCAAGACAACATTATATATGCA |
| | TTGGTATCA |
| 39. HOS3-uptag | ATTCGACGATATAATATGGATGTCCACGAGGTCTCT |
| | GAGATCATGCCATTCAAGCCCGTACGCTGCAGGTCG |
| | AC |
| 40. HOS3-dntag | ATATATGCATTGGTATCACGGTGTCGGTCTCGTAGA |
| | GGCGATGATGGCATTTACTATCGATGAATTCGAGC |
| | TCG |
| 41. KanB | CTGCAGCGAGGAGCCGTAAT |
| 42. KanC | TGATTTTGATGACGAGCGTAAT |
| 43. Kan-up | CGTACGCTGCAGGTCGACGG |
| 44. Kan-Dn | ATCGATGAATTCGAGCTCGTTT |

### Example 2

### Antifungal Compound Sensitivity of Deletion Mutant Strains

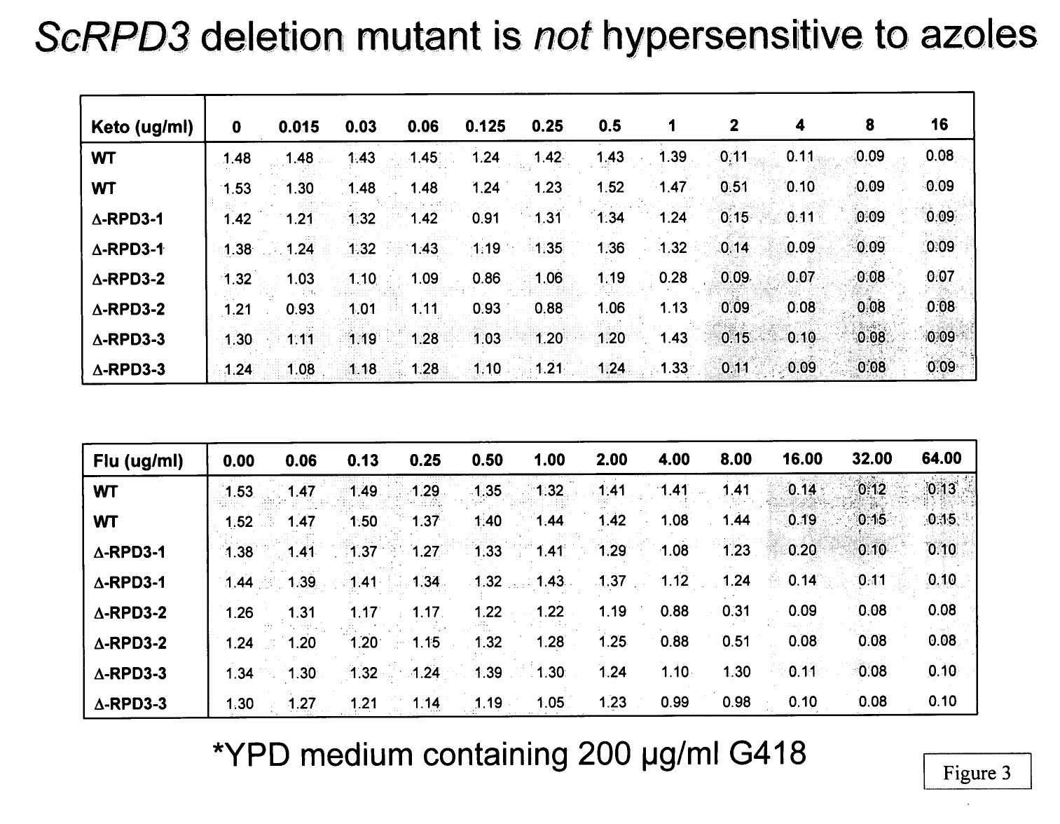

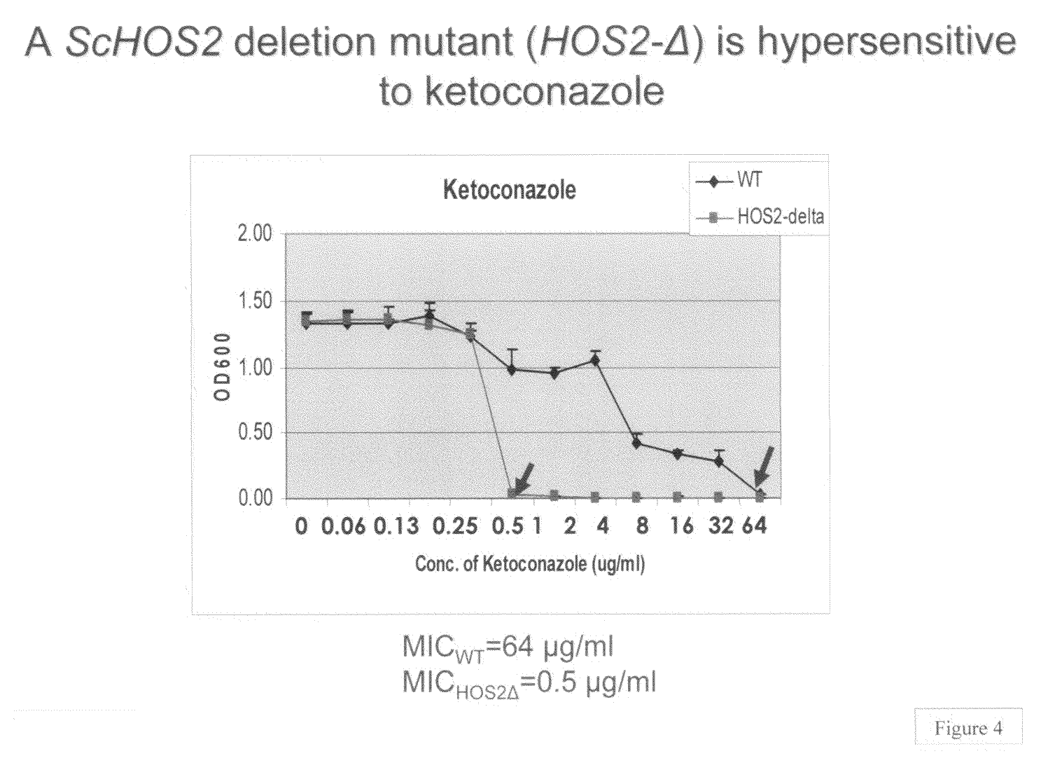

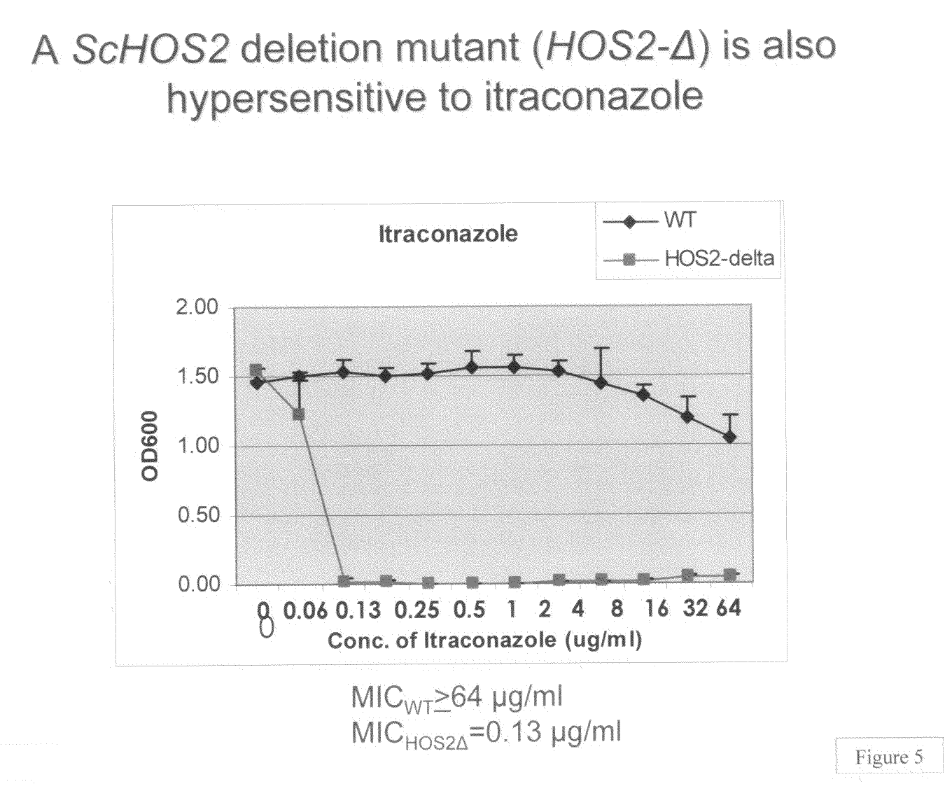

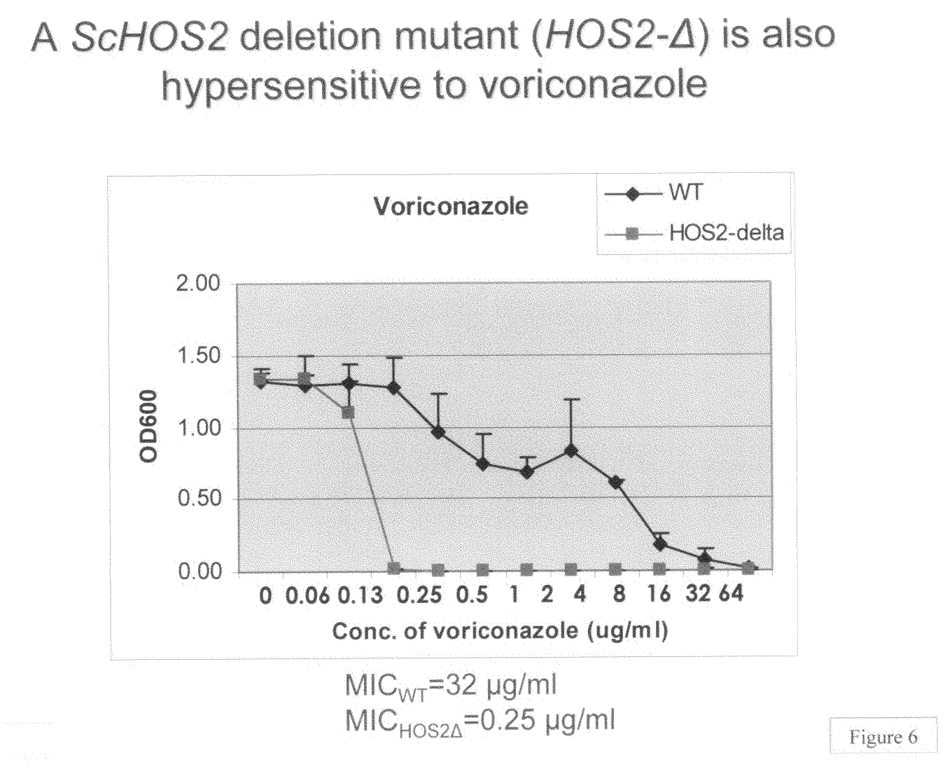

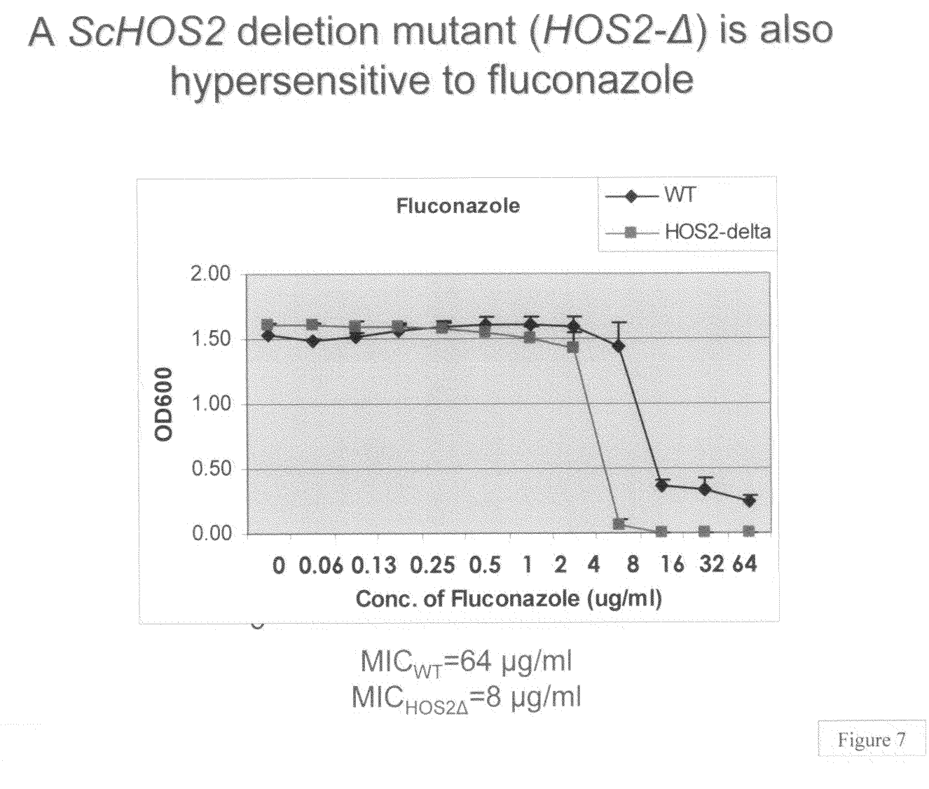

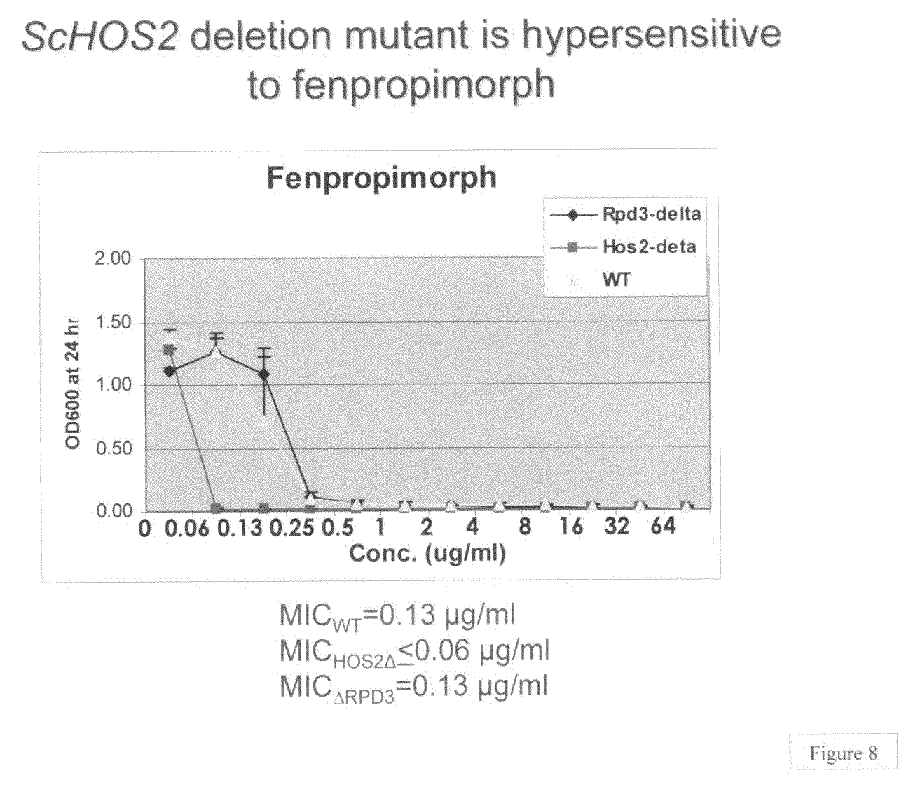

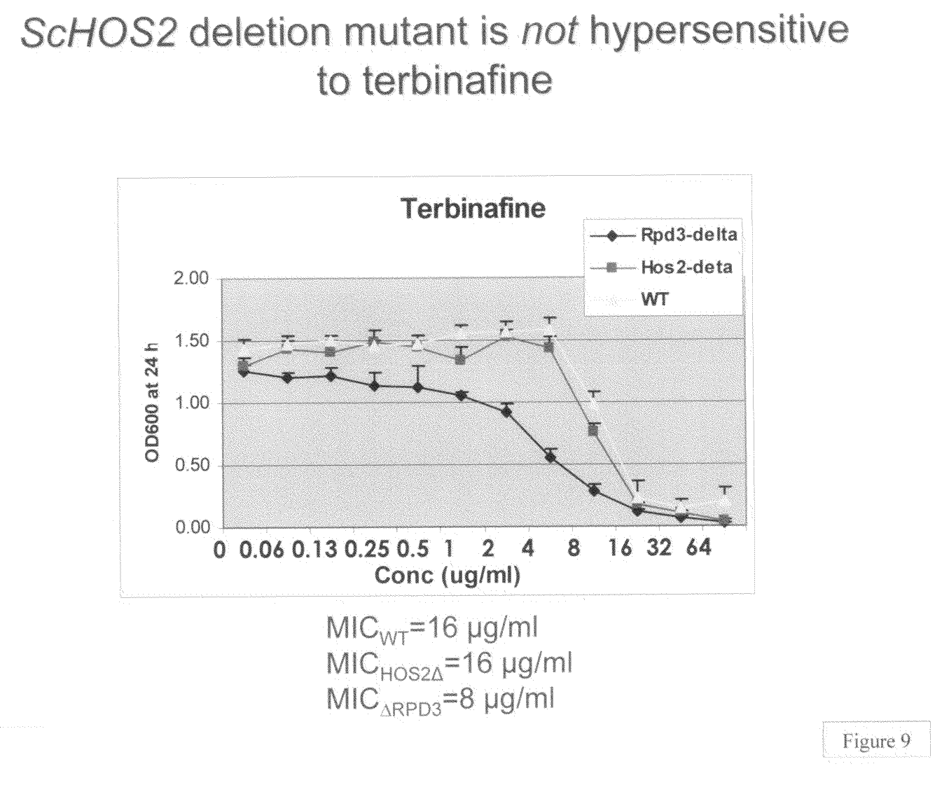

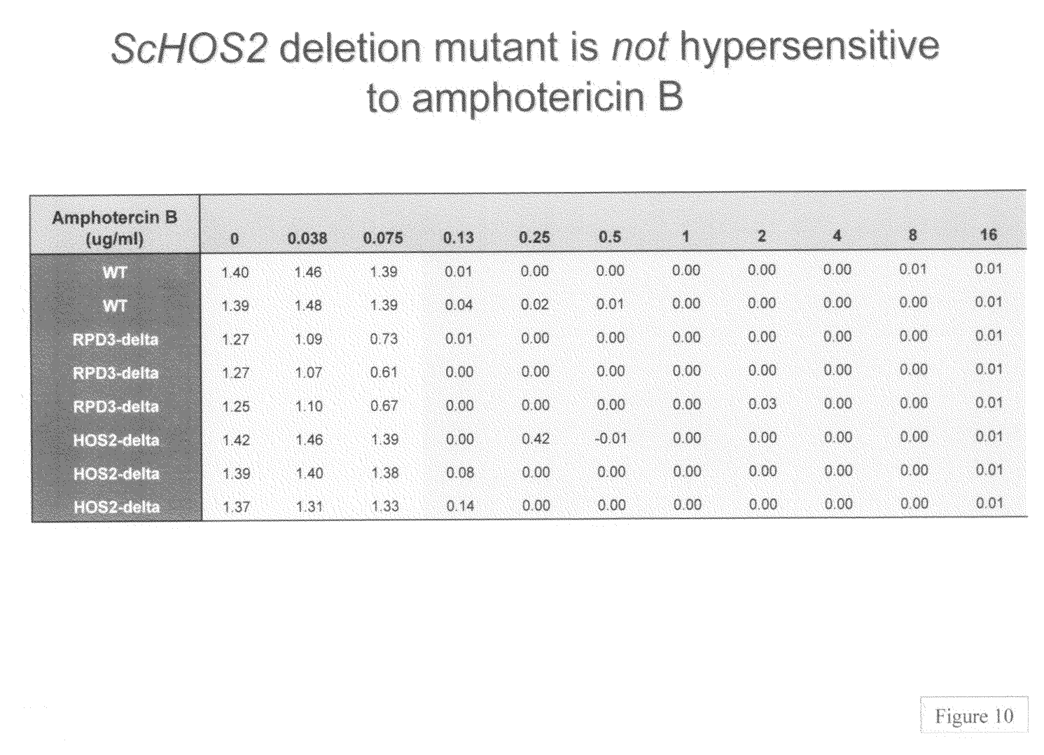

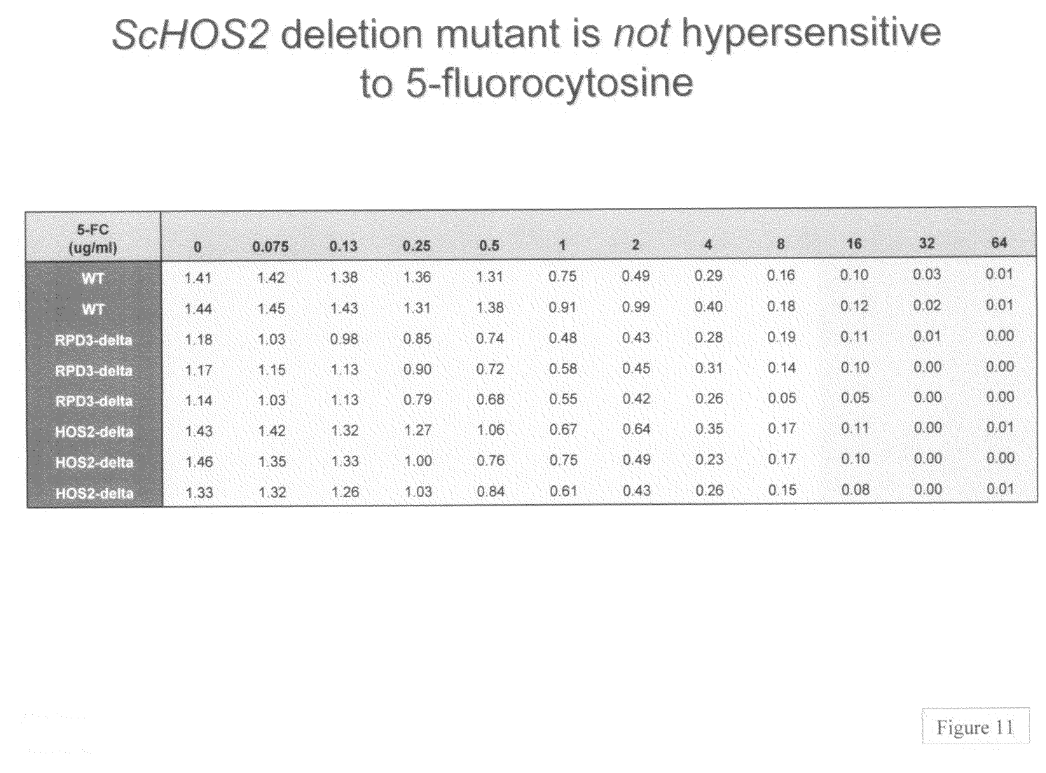

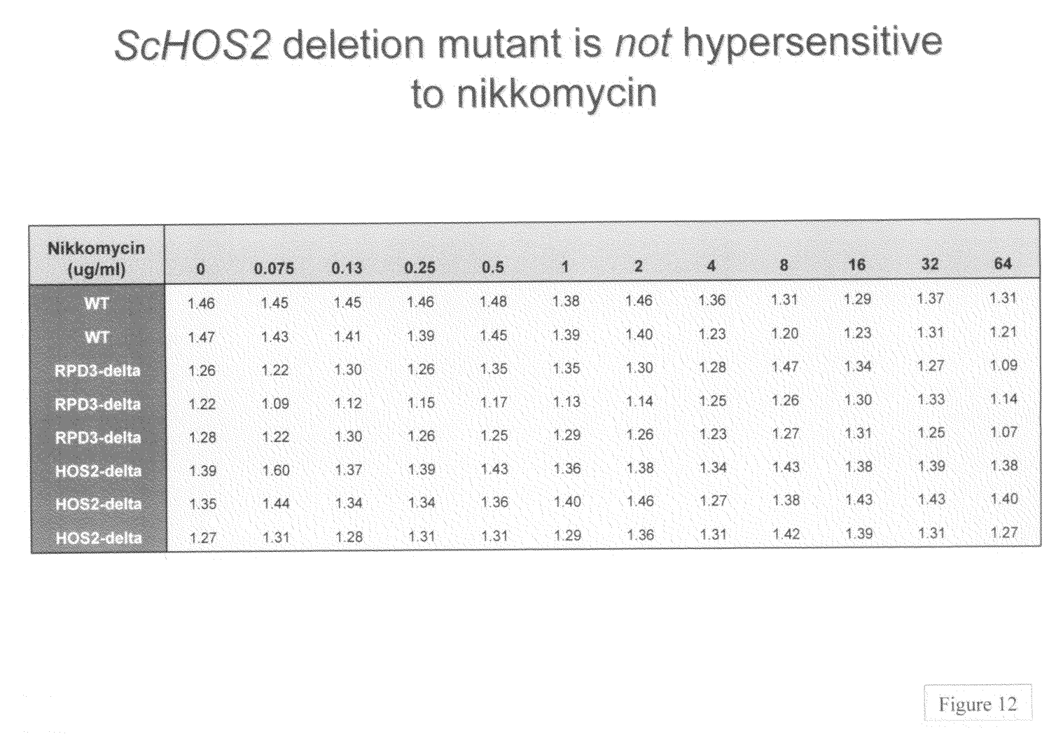

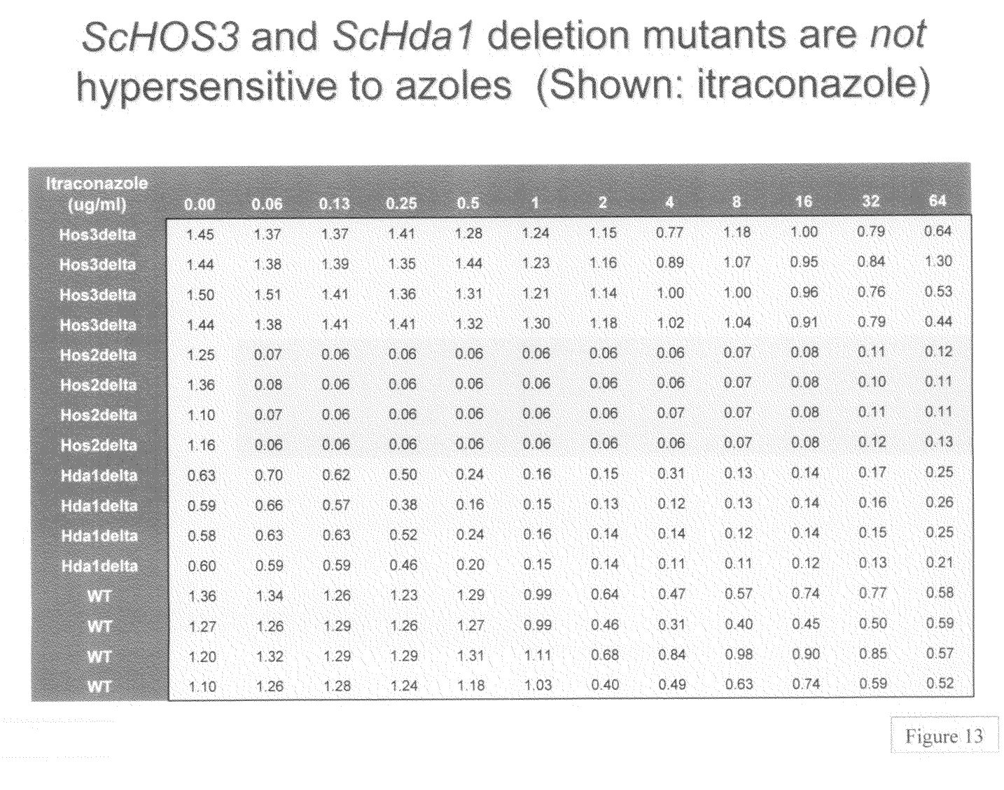

Standard serial dilution techniques were used to determine sensitivity of yeast deletion strains to various antifungal agents. FIG. 3 shows that a ScRPD3 deletion mutant is not hypersensitive to ketoconazole or fluconazole. FIG. 4 shows that a ScHOS2 deletion mutant is hypersensitive to ketoconazole. FIG. 5 shows that a ScHOS2 deletion mutant is hypersensitive to itraconazole. FIG. 6 shows that a ScHOS2 deletion mutant is hypersensitive to voriconazole. FIG. 7 shows that a ScHOS2 deletion mutant is hypersensitive to fluconazole. FIG. 8 shows that a ScHOS2 deletion mutant is hypersensitive to fenpropimorph. FIG. 9 shows that a ScHOS2 deletion mutant is not hypersensitive to terbinazine. FIG. 10 shows that a ScHOS2 deletion mutant is not hypersensitive to Amphotericin B. FIG. 11 shows that a ScHOS2 deletion mutant is not hypersensitive to 5-fluorocytosine. FIG. 12 shows that a ScHOS2 deletion mutant is not hypersensitive to nikkomycin. FIG. 13 shows that ScHOS3 and ScHda1 deletion mutants are not hypersensitive to itraconazole. ScHOS3 and ScHda1 deletion mutants were also not hypersensitive to other azoles (data not shown).

Construction and Expression of Recombinant His₆V₅-Tagged Hos2p from S. cerevisiae

The ORF encoding YGL194C/HOS2 from S. cerevisiae (residues 1-452 upstream the stop codon) was PCR-amplified with Taq DNA polymerase (Invitrogen) using genomic DNA isolated from the BY4742 strain (ATCC201389) and the following primers: MYG1213 (ScHOS2-Forward) 5′-TAATGTCTGGAACATTTAGTTATGATGTGAAAACAAAG-3′ and MYG1212 (ScHOS2-Reverse) 5′-TGAAAAGGCAATCAATCCACTGTTTTCTTTTTCCAT-3′. PCR amplification was performed as follows: 2 min. denaturation at 94° C. followed by a touch-up PCR (1 min. denaturation at 94° C., 1 min. hybridization at 43-53° C. with an increase of 3° C. between each cycle, 1.5 min.” amplification at 72° C.), followed by 30 cycles composed of 1 min. denaturation at 94° C., 1 min hybridization at 53° C., 1.5 min amplification at 72° C. A 1.4 kb PCR product was gel purified and used for cloning into the pYES2.1/NV5-His-TOPO vector TOPO and subsequent transformation of TOP 10 One Shot Chemically Competent E. coli using the pYES2.1 TOPO TA Expression Kit (Invitrogen, cat. # K4150-01) according to the manufacturer's instructions. The presence of the HOS2.V5-His6 insert was analyzed by PCR in transformants using primers MYG1213 and MYG1212 in combination with primers V5-Cterm-Reverse and GAL 1-Forward, respectively, provided with the pYES2.1 TOPO TA Expression Kit. Plasmids pYES2.1.HOS2.V5His6 isolated from 2 bacterial clones (pYES2.1.HOS2.V5His6 # 2 and # 7) were used to transform the S. cerevisiae BY4742 (MATα his3delta1 leu2delta0 lys2delta0 ura3delta0, ATCC 201389) strain and the Δhos2 isogenic deletion strain (generated in-house) using the S.c. EasyComp Transformation Kit (Invitrogen, cat. # 5050-01) according to the manufacturer's instructions. For each plasmid used for the transformation, whole cell extracts were prepared from three individual clones of BY4742/pYES2.1.HOS2.V5His6 and Δhos2/pYES2.1.HOS2.V5His6 were tested for the expression of the recombinant Hos2p.V5His6 protein using anti-V5-HRP antibodies (Invitrogen cat. # R961-25) following induction by galactose for 24 h. Strains BY4742/pYES2.1.HOS2.V5His6 #7 and Δhos2/pYES2.1.HOS2.V5His6 #7 were kept for further studies.

### Induction and Purification of Recombinant Hos2 Protein

The induction of Hos2p.V5His6 expression by galactose was performed using either glucose or raffinose as the carbon source (pYES2.1 TOPO TA Expression Kit Manual, Invitrogen, cat. # K4150-01), Cell lysates were prepared from spheroplasts that had been generated from strains BY4742/pYES2.1.HOS2.V5His6 # 7 and Δhos2/pYES2.1.HOS2.V5His6 #7 grown in the presence or in the absence of galactose. Spheroplasts were prepared from cells that were grown in YEPD (BY4742, Δhos2), SC-URA+2% glucose or raffinose or SC-URA+1% glucose or raffinose+1% galactose (BY4742/pYES2.1.HOS2.V5His6 # 7 and Δhos2/pYES2.1.HOS2.V5His6 #7) by the lyticase method of Franzusoff et al. (1991). The collection of fractions containing or not Hos2pV5His6 was performed by IMAC using the H is GraviTrap Affinity Columns (GE Healthcare cat. # 11-033-99). Binding buffer was as recommended by the manufacturer, whereas three different elution buffers were used sequentially: 20 mM sodium phosphate+500 mM NaCl+100 mM, 250 mM or 500 mM imidazole, pH 7.4. Fractions were further desalted using PD-10 (GE Healthcare, cat. # 17-0851-01) and NAP-5 (GE Healthcare, cat. # 17-0853-02) desalting columns. Fractions that were eluted with the three different buffers containing 100, 250 or 500 mM imidazole, pH 7.4 and desalted were assayed for HDAC activity (see below). Only the corresponding fractions containing significant intrinsic HDAC activity (20 mM sodium phosphate+500 mM NaCl+250 mM imidazole) were kept for further studies.

### HDAC Assay

Fractions containing the recombinant Hos2p.V5His6 were incubated for 2 h with or without HDAC inhibitor (0-20 μg/ml). The HDAC activity that was specifically associated with the recombinant Hos2p.V5His6 expressed following galactose induction was determined assuming that

Fluo_{Hos2p.V5His6 specific}=Fluo_{+galactose}−Fluo_{-galactose}

where
Fluo_{+galactose}=HDAC activity associated with all HDACs copurified along with Hos2p.V5His6
Fluo_{+glucose}=HDAC activity associated with all HDACs copurified in the absence of Hos2p.V5His6

The results of the inhibition assays are summarized in Table 1. As can be seen, purified Hos2 protein was inhibited by more than 60% with 5 ug/ml compound 4.

TABLE 1

| | % inhibition at 5 μg/ml | IC50 (ug/ml) |
|---|---|---|
| Source of Hos2 protein | Compound 4 | Compound 4 |
| S. cerevisiae | 60.7 | ~4 |
| BY4742/HOS2.V5His6 grown in | | |
| the presence of glucose (GAL1 | | |
| promoter repressed) or galactose | | |
| (GAL1 promoter induced) | | |
| S. cerevisiae | 55.2 | ~4.5 |
| Δhos2/HOS2.V5His6 grown in | | |
| the presence of glucose (GAL1 | | |
| promoter repressed) or galactose | | |
| (GAL1 promoter induced) | | |
| S. cerevisiae | 61.2 | ~3 |
| BY4742/HOS2.V5His6 grown in | | |
| the presence of raffinose (GAL1 | | |
| promoter repressed) or galactose | | |
| (GAL1 promoter induced) | | |

### Recombinant ScHos2 Protein Helps Overcome Hos2 Deletion Mutant Susceptibility to Itraconazole

### MIC

Minimum inhibitory concentration (MIC) testing was performed according to CLSI M27-A2 method for yeast testing using CSM-URA medium (Complete Synthetic Medium minus Uracile) containing either 2% raffinose or 1% raffinose+1% galactose as the carbon source. Itraconazole concentrations ranged from 0.01-110 g/mL. Log-phase cells grown in CSM-URA+2% raffinose or CSM-URA+1% raffinose+1% galactose were inoculated at 5×10³ cells/ml. The MIC₈₀ of itraconazole was determined after 72 hours growth at 30° C.

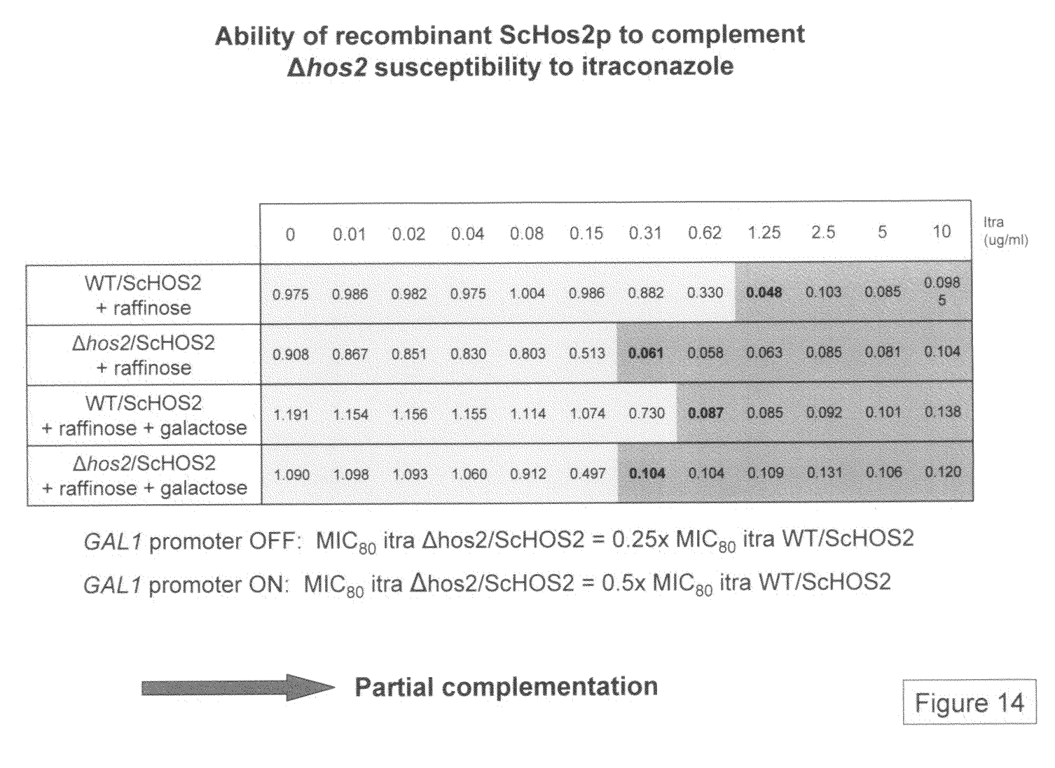

As shown in FIG. 14, there was a 4-fold difference in sensitivity to itraconazole between WT/ScHOS2 and Δhos2/ScHOS2, when they were grown in the presence of raffinose, while the difference in sensitivity was only 2-fold when expression of Hos2 was induced by galactose.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for screening a compound for potential antifungal activity and/or to screen for a compound with the ability to potentiate an antifungal agent, comprising providing a HDAC mutant fungal strain, the mutation resulting in loss of function of the protein encoded thereby and contacting the HDAC mutant fungal strain with the compound, wherein sensitivity of the HDAC mutant fungal strain to the test compound identifies the compound as having potential antifungal activity, said activity potentiated by inhibition of an HDAC gene, or homolog thereof, or an HDAC gene product, or homolog thereof.

2. A method for screening a compound for potential antifungal activity and/or to screen for a compound with the ability to potentiate an antifungal agent, comprising providing a fungal HDAC protein or homolog thereof, or active fragment thereof and contacting the protein or homolog thereof or active fragment thereof with the compound, wherein inhibition of HDAC activity of the protein or homolog thereof or active fragment thereof identifies the compound as having potential antifungal activity or the ability to potentiate the antifungal activity of an antifungal agent.

3. A method for testing antifungal compounds for potential synergy with a fungal HDAC inhibitor, the method comprising providing a HOS2 knockout fungal strain, treating the knockout mutant strain with a test compound, determining the sensitivity of the HOS2 knockout fungal strain to the test compound, providing one or more control fungal strain selected from the group consisting of a wild-type strain, an RPD3 knockout mutant strain, an HDA1 knockout mutant strain, a HOS1 knockout mutant strain, a HOS3 knockout mutant strain and a SIR2 knockout mutant strain, treating the one or more control fungal strain with the test compound, determining the sensitivity of the one or more test strain to the test strain, and comparing the sensitivity of the HOS2 knockout strain to the test compound with the sensitivity of the one or more control strain to the test compound, wherein the test compound is considered to be an antifungal compound having synergy with a HOS2 inhibitor if the HOS2 knockout mutant strain is more sensitive to the test compound than the one or more control strain.

4. A method for inhibiting fungal growth, comprising inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

5. A method for inhibiting trailing growth of a fungus in the presence of an antifungal compound, comprising inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

6. A method for inhibiting resistance of a fungus to an antifungal compound, comprising inhibiting the expression of fungal HOS2 or a homolog thereof, or inhibiting the activity of a gene product thereof, and treating the fungus with the antifungal compound, the activity of which is potentiated by inhibition of expression of the HOS2 or a homolog thereof or inhibition of activity of a gene product thereof.

7. A method for inhibiting survival of a fungus in the presence of an antifungal compound, comprising inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

8. A method for reducing ability of a fungus to resist antifungal activity of an antifungal compound, comprising inhibiting the expression of fungal HOS2 or a homolog thereof, or inhibiting the activity of a gene product thereof, and treating the fungus with an antifungal compound, the activity of which is potentiated by inhibition of expression of the HOS2 or a homolog thereof or inhibition of activity of a gene product thereof.

9. A method for enhancing fungal sensitivity to an antifungal compound, the activity of which is potentiated by inhibition of expression of HOS2 or a homolog thereof or inhibition of activity of a HOS2 gene product or homolog thereof, comprising inhibiting the expression of fungal HOS2 or a homolog thereof, or inhibiting activity of a gene product thereof.

10. A method for potentiating the antifungal activity of an antifungal compound, the activity of which is potentiated by inhibition of expression of HOS2 or a homolog thereof or inhibition of activity of a HOS2 gene product, or homolog thereof, comprising inhibiting the expression of fungal HOS2 or a homolog thereof, or inhibiting activity of a gene product thereof.

11. A method for therapeutically treating an organism having a fungal infection, comprising inhibiting the expression of fungal HOS2 or a fungal homolog thereof, or inhibiting the activity of a gene product thereof, in an organism having a fungal infection, and treating the organism with an antifungal compound, the activity of which is potentiated by inhibition of expression of the HOS2 or a fungal homolog thereof or inhibition of activity of a gene product thereof.

12. A method for preventing a fungal infection in an organism, comprising treating the organism with an antifungal compound, the activity of which is potentiated by inhibition of expression of the HOS2 or a homolog thereof or inhibition of activity of a gene product thereof, and profilactively treating the organism with a HOS2 and/or a Hos2 inhibitor compound.

13. A method for identifying a potentiator of an antifungal compound comprising administering to a fungal strain a test compound, determining whether the test compound inhibits expression of HOS2, or a homolog thereof, and identifying the test compound as a potentiator of an antifungal compound If the test compound inhibits HOS2, or the homolog thereof.

14. An inhibitor of Hos2, a homolog thereof or fungal HOS2, or a homolog thereof.

15. A composition comprising the inhibitor of claim 14 and a pharmaceutically acceptable carrier.

16. A method of inhibiting Hos2, or a homolog thereof, comprising contacting the Hos2, or a homolog thereof, with the inhibitor of claim 14.

17. A method of inhibiting HOS2, or a homolog thereof, comprising contacting the HOS2, or a homolog thereof, with the inhibitor of claim 14.

18. The inhibitor of claim 14, wherein the inhibitor of Hos2, or the homolog thereof, is a hydroxymate compound.
